(19) United States
(12) Patent Application Publication
(10) Pub. No.: US 2020/0157568 A1
(43) Pub. Date: May 21, 2020

(54) EXPRESSION VECTORS AND RELATED METHODS OF DELIVERY OF Na/K ATPASE/Src RECEPTOR COMPLEX ANTAGONISTS

(71) Applicant: MARSHALL UNIVERSITY RESEARCH CORPORATION, Huntington, WV (US)

(72) Inventors: Zijian XIE, Saline, MI (US); Komal SODHI, Huntington, WV (US); Nader ABRAHAM, Valhalla, NY (US); Rebecca MARTIN, Huntington, WV (US); Joseph SHAPIRO, Huntington, WV (US)

(21) Appl. No.: 16/615,652

(22) PCT Filed: May 25, 2018
(86) PCT No.: PCT/US2018/034716

§ 371 Date: Nov. 21, 2019

Related U.S. Application Data
(60) Provisional application No. 62/511,541, filed on May 26, 2017.

(51) Int. Cl. C12N 15/86; C12N 9/14

(52) U.S. Cl. CPC C12N 15/86; C12N 9/14; C12N 2840/007; C12Y 306/03009; C12N 2740/15043

(57) ABSTRACT

Expression vectors are provided that comprise a nucleic acid sequence encoding a polypeptide antagonist of a Na/K ATPase/Src receptor complex. The nucleic acid encoding the polypeptide antagonist is operatively linked to a promoter for expressing the polypeptide antagonist in a specific cell or tissue. Viral particles, target cells, and pharmaceutical compositions are also provided and include the expression vectors. Methods of treating a Src-associated disease is further provided and includes administering the expression vectors encoding the polypeptide antagonist of the Na/K ATPase/Src receptor complex to a subject in need thereof.

### RELATED APPLICATIONS

This application claims priority from U.S. Provisional Application Ser. No. 62/511,541, filed May 26, 2017, the entire disclosure of which is incorporated herein by this reference.

### TECHNICAL FIELD

The presently-disclosed subject matter relates to expression vectors and methods for delivering Na/K ATPase/Src receptor complex antagonists. In particular, certain embodiments of the present invention relate to expression vectors and related methods for delivery of Na/K ATPase/Src receptor complex antagonists to specific cells and tissues, as well as methods for using such vectors to treat a Src-associated disease.

### BACKGROUND

The Na/K-ATPase enzyme is ubiquitously expressed in most eukaryotic cells and helps maintains the trans-membrane ion gradient by pumping Na⁺ out and K⁺ into cells. The Na/K-ATPase interacts directly with Src via at least two binding motifs: one being between the CD2 of the α1 subunit and Src SH2; and, the other involving the third cytosolic domain (CD3) and Src kinase domain. The formation of this Na/K-ATPase and Src complex serves as a receptor for ouabain to provoke protein kinase cascades. Specifically, binding of ouabain to Na/K-ATPase will disrupt the latter interaction, and then result in assembly and activation of different pathways including ERK cascades, PLC/PKC pathway and ROS production. Moreover, this interaction keeps Src in an inactive state. Thus, the Na/K-ATPase functions as an endogenous negative Src regulator. See also International Patent Application Nos. WO 2008/054792 and WO 2010/071767, which are both incorporated herein by reference.

The activation of these signaling pathways eventually leads to changes in cardiac and renal functions, stimulation of cell proliferation and tissue fibrosis, protection of tissue against ischemia/reperfusion injury, inhibition of cancer cell growth, and more. Src and ROS are also involved in the induction of VEGF expression. While many known Src and Src family kinase inhibitors are developed as ATP analogs that compete for ATP binding to these kinases, such Src inhibitors lack pathway specificity. Accordingly, compositions and methods for targeting cells and tissues for improved treatment of a wide variety of conditions related to Na/K-ATPase-Src interactions would be highly desirable and beneficial.

### SUMMARY

The presently-disclosed subject matter meets some or all of the above-identified needs, as will become evident to those of ordinary skill in the art after a study of information provided in this document. This summary describes several embodiments of the presently-disclosed subject matter, and in many cases lists variations and permutations of these embodiments.

This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this summary does not list or suggest all possible combinations of such features.

The presently-disclosed subject matter includes expression vectors and methods for delivering Na/K ATPase/Src receptor complex antagonists. In particular, certain embodiments of the present invention relate to expression vectors and related methods for delivery of Na/K ATPase/Src receptor complex antagonists to specific cells and tissues, as well as methods for using such vectors to treat a Src-associated disease. In some embodiments, an expression vector is provided that comprises a nucleic acid sequence encoding a polypeptide antagonist of a Na/K ATPase/Src receptor complex. In some embodiments, the nucleic acid encoding the polypeptide antagonist is operatively linked to a promoter for expressing the polypeptide antagonist in a specific cell or tissue. In some embodiments, the polypeptide anatagonist comprises the sequence of SEQ ID NO: 1, or a fragment and/or variant thereof. In some embodiments, the nucleic acid encoding the polypeptide antagonist comprises the sequence of SEQ ID NO: 5, or a fragment and/or variant thereof. In some embodiments, the promoter is selected from an adiponectin promoter, an albumin promoter, a melanin promoter, a vonWillebrand factor promoter, an alpha myosin heavy chain promoter, a SGLT2 promoter, a MyoD promoter, a glial fibrillary acidic protein (GFAP) promoter, and a synapsin 1 (SYN1) promoter. In certain embodiments, the promoter is liver-specific, endothelial cell-specific, or adipose cell-specific.

In some embodiments of the presently-disclosed subject matter, the expression vectors are in the form of a viral vector such as, in certain embodiments, a lentivirus vector. In that regard, in some embodiments, viral particles that include the expression vectors described herein are also provided along with target cells that include the expression vectors of the presently-disclosed subject matter. In some embodiments, the target cell is mammalian, such as a mouse cell or a human cell. In some embodiments, the target cell is an adipose cell, a liver cell, or an endothelial cell.

Further provided, in some embodiments, are pharmaceutical compositions. In some embodiments, a pharmaceutical composition is provided that comprises an expression vector of the presently-disclosed subject matter and a pharmaceutically acceptable vehicle, carrier, or excipient.

Still further provided, in some embodiments, are methods of treating a Src-associated disease. In some embodiments, a method of treating a Src-associated disease is provided that comprises administering the expression vector of the presently-disclosed subject matter to a subject in need thereof. In some embodiments, the Src-associated disease is selected from the group consisting of vascular disease, cardiovascular disease, heart disease, prostate cancer, breast cancer, neuroblastoma, cardiac hypertrophy, tissue fibrosis, congestive heart failure, ischemia/reperfusion injury, osteoporosis, retinopathy, and obesity. In some embodiments, the Src-associated disease is cardiovascular disease, and the cardiovascular disease is uremic cardiomyopathy. In some embodiments, the Src-associated disease is obesity.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 is a schematic diagram showing a lentiviral expression vector made in accordance with the presently-disclosed subject matter, and encoding a polypeptide antagonist of a Na/K ATPase/Src receptor complex (NaKtide; SEQ ID NO: 1) under the control of an adiponectin promoter.

FIG. 2 is a schematic diagram showing another lentiviral expression vector made in accordance with the presently-disclosed subject matter, and encoding an enhanced green flourescent protein (eGFP) under the control of an adiponectin promoter.

FIG. 3 includes fluorescent microscopy images of 3T3-L1 cells that were transduced with increasing Multiplicity of Infection (MOI) of the expression vectors shown in FIGS. 1 and 2.

FIG. 4 includes images and a graph showing oil red O staining in 3T3-L1 cells that were transduced with increasing Multiplicity of Infection (MOI) of the expression vectors shown in FIGS. 1 and 2.

FIGS. 5A-5D includes immunofluorescence staining of adipose tissue (FIG. 5A), liver tissue (FIG. 5B), heart tissue (FIG. 5C), and kidney tissue (FIG. 5D) in C57Bl6 mice administered the expression vectors shown in FIGS. 1 and 2.

FIG. 6 is a schematic diagram showing a lentiviral expression vector made in accordance with the presently-disclosed subject matter, and encoding a polypeptide antagonist of a Na/K ATPase/Src receptor complex (NaKtide; SEQ ID NO: 1) under the control of an albumin promoter.

FIG. 7 is a schematic diagram showing another lentiviral expression vector made in accordance with the presently-disclosed subject matter, and encoding an enhanced green flourescent protein (eGFP) under the control of an adiponectin promoter.

FIGS. 8A-8B includes images showing immunohistochemistry staining of liver tissue (FIG. 8A) and adipose tissue (FIG. 8B) of C57Bl6 mice administered the expression vectors shown in FIGS. 6 and 7.

FIG. 9 includes images and a graph showing the effect of lentiviral transfected NaKtide (SEQ ID NO: 1 and 6) on body weight in mice fed a high-fat diet, where NaKtide administered via lentivirus resulted in a significant reduction in the amount of weight gained by C57Bl6 mice fed a high-fat diet compared to their control chow counterparts, where injections were given at Week 0 and Week 2 in mice fed a western diet for 12 weeks, and where there was no significant change in food intake among the groups (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIGS. 10A-10B includes images and graphs showing the effect of lentiviral transfected NaKtide on visceral and subcutaneous fat content in C57Bl6 mice fed a Western diet (WD) for 12 weeks, where the mice were injected with NaKtide at Week 0 and 2, and where administration of NaKtide to mice fed the high-fat diet significantly reduced visceral (FIG. 10B) and subcutaneous (FIG. 10A) fat content as compared to high-fat diet-fed animals (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIGS. 11A-11D include graphs showing the effect of lentiviral transfected NaKtide on metabolic and inflammatory cytokines in mice fed a western diet, where C57Bl6 mice fed a Western diet (WD) for 12 weeks were injected with NaKtide at Week 0 and 2, where administration of NaKtide to mice fed a high-fat diet significantly reduced changes in oral glucose tolerance test (GTT; FIG. 11A), and where inflammatory markers TNFα (FIG. 11B), IL-6 (FIG. 11D), and MCP-1 (FIG. 11C) also showed significant reduction in mice administered NaKtide as compared to mice fed a western diet with no treatment (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIGS. 12A-12E include graphs showing the effect of adipocyte-specific NaKtide expression on leptin (FIG. 12A), systolic blood pressure (FIG. 12B), oxygen consumption (FIG. 12C), activity (FIG. 12D), and energy expenditure (FIG. 12E) in mice fed a western diet, where NaKtide administered via lentivirus resulted in a significant increases in plasma leptin concentration of mice fed western diet, which was decreased upon treatment with lenti-adiponectin-NaKtide, where mice fed a western diet showed significant increases in systolic blood pressure, ameliorated by lenti-adiponectin-NaKtide, and where oxygen consumption, activity, and energy expenditure were all significantly increased in mice treated with lenti-adiponectin-NaKtide compared to western diet fed animals (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIGS. 13A-13D include images and graphs showing the effect of adipocyte specific NaKtide expression on adipogenesis related proteins (FIG. 13A), Na/K-ATPase signaling markers (FIG. 13B), and brown fat marker PGC1α (FIG. 13C) in mice fed a western diet, where NaKtide administered via lentivirus resulted in a significant increase in markers associated with adipogenesis, and phosphorylated Src, where expression of the alpha 1 subunit of the Na/K-ATPase was decreased in mice fed a western diet, and increased in mice treated with lenti-adiponectin-NaKtide, where brown fat marker PGC1α was significantly decreased in western diet fed mice, and increased in the visceral fat of mice treated with lenti-adiponectin-NaKtide, and where protein carbonylation (FIG. 13D) was increased in mice fed a western diet and attenuated in mice treated with lenti-adiponectin-NaKtide (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIG. 14 includes images and graphs showing the effect of adipocyte specific NaKtide expression on adipocyte size and number in visceral fat in mice fed a western diet, where mice fed a western diet had significantly less adipose cells in visceral fat compared to control animals, as well as a significant increase in the area of the cells present, and where lenti-adiponectin-NaKtide decreased the area of adipose cells and increased the amount of cells present (results are means±SE, n=12 to 14 per group; *P<0.05 versus Control, # P<0.05 versus Control+GFP+NaKtide, +P<0.05 versus Western diet, &P<0.05 versus Western diet Diet+GFP).

FIGS. 15A-15G include images and graphs showing the effectiveness of NaKtide in C57BL6 PNx model (FIG. 15A) with immunofluorescence staining of NaKtide in adipose and liver tissue; oxidative stress using TBARS assessment (FIG. 15B), glucose tolerance test (FIG. 15C) level of cytokines, IL-6 and MCP-1 respectively (FIGS. 15D-15E), and RT-PCR analyses of PGC1α and Sirt3 expressions respectively (FIGS. 15F-15G) (** p<0.01 vs. Sham; ## p<0.01 vs. PNx; (n=6)).

FIGS. 16A-16E includes graphs and diagrams showing the effect of NaKtide on (FIG. 16A) HW/BW ratio, (FIG. 16B) cardiac fibrosis measured with Sirius Red staining, (FIG. 16C) hematocrit level, (FIG. 16D) plasma creatinine level, and (FIG. 16E) cardiac hypertrophy, assessed with transthoracic echocardiography measurements, including, LVM, left ventricular mass; EF, ejection fraction; MPI, myocardial performance index; RWT, relative wall thickness (values are means±SEM. ** p<0.01 vs. Sham; ## p<0.01 vs. PNx; ++p<0.01 vs. PNx+WD; (n=6)).

FIGS. 17A-17D include graphs showing the effectiveness of lenti-adiponectin-NaKtide in C57BL6 PNx model, and including RT-PCR analyses of inflammatory and apoptotic markers, (FIG. 17A) TNF-α, (FIG. 17B) IL-6, (FIG. 17C) Casp7, and (FIG. 17D) Bax (values are means±SEM. ** p<0.01 vs. Sham; ## p<0.01 vs. Sham+WD; ++p<0.01 vs. PNx+WD; $$p<0.01 vs. PNx (n=6)).

FIGS. 18A-18D include graphs showing the effectiveness of lenti-adiponectin-NaKtide in C57BL6 PNx model, and including RT-PCR analyses of markers of mitochondrial biogenesis, (FIG. 18A) Leptin, (FIG. 18B) F4/80, (FIG. 18C) Sirt3 and (FIG. 18D) PGC1α, where values are means±SEM. ** p<0.01 vs. Sham; ## p<0.01 vs. Sham+WD; ++p<0.01 vs. PNx+WD; $$p<0.01 vs. PNx (n=6).

FIG. 19 is a schematic diagram showing a lentiviral expression vector made in accordance with the presently-disclosed subject matter, and encoding a polypeptide antagonist of a Na/K ATPase/Src receptor complex (NaKtide; SEQ ID NO: 1) under the control of an alpha myosin heavy chain promoter.

FIG. 20 is a schematic diagram showing a lentiviral expression vector made in accordance with the presently-disclosed subject matter, and encoding a polypeptide antagonist of a Na/K ATPase/Src receptor complex (NaKtide; SEQ ID NO: 1) under the control of an SGLT2 promoter.

FIG. 21 is a schematic diagram showing a lentiviral expression vector made in accordance with the presently-disclosed subject matter, and encoding a polypeptide antagonist of a Na/K ATPase/Src receptor complex (NaKtide; SEQ ID NO: 1) under the control of an MyoD promoter.

FIG. 22 is a schematic diagram showing a lentiviral expression vector made in accordance with the presently-disclosed subject matter, and encoding a polypeptide antagonist of a Na/K ATPase/Src receptor complex (pNaKtide; SEQ ID NO: 5) under the control of an glial fibrillary acidic protein (GFAP) promoter.

FIG. 23 is a schematic diagram showing a lentiviral expression vector made in accordance with the presently-disclosed subject matter, and encoding a polypeptide antagonist of a Na/K ATPase/Src receptor complex (pNaKtide; SEQ ID NO: 5) under the control of a synapsin I (SYN1) promoter.

### BRIEF DESCRIPTION OF THE SEQUENCE LISTING

The following is a brief description of the Sequence Listing that is attached hereto and is hereby incorporated by reference in its entirety.

SEQ ID NO: 1 is an amino acid sequence encoding an embodiment of a polypeptide in accordance with the presently-disclosed subject matter (NaKtide);

SEQ ID NO: 2 is an amino acid sequence encoding a TAT cell penetrating peptide;

SEQ ID NO: 3 is an amino acid sequence encoding a penetratin (AP) cell penetrating peptide; and

SEQ ID NO: 4 is an amino acid sequence encoding the N-terminal poly-lysine domain of the α1 subunit of Na/K-ATPase (A1N).

SEQ ID NO: 5 is another amino acid sequence of an embodiment of a polypeptide in accordance with the presently-disclosed subject matte (pNaKtide).

SEQ ID NO: 6 is a nucleic acid sequence of a lentivirus gene expression vector encoding a green fluorescent protein (GFP) and a polypeptide antagonist of a Na/K ATPase/Src receptor complex (nucleotide position 7398-7460; NaKtide; SEQ ID NO: 1) operably connected to an adiponectin promoter (nucleotide position 1959-7367).

SEQ ID NO: 7 is a nucleic acid sequence of a lentivirus gene expression vector encoding a green fluorescent protein (GFP) and a polypeptide antagonist of a Na/K ATPase/Src receptor complex (nucleotide position 4325-4387; NaKtide; SEQ ID NO: 1) operably connected to an albumin promoter (nucleotide position 1959-4294).

SEQ ID NO: 8 is a nucleic acid sequence of a lentivirus gene expression vector encoding a green fluorescent protein (GFP) and a polypeptide antagonist of a Na/K ATPase/Src receptor complex (nucleotide position 7453-7515; NaKtide; SEQ ID NO: 1) operably connected to an alpha myosin heavy chain promoter (7453-7515).

SEQ ID NO: 9 is a nucleic acid sequence of a lentivirus gene expression vector encoding a green fluorescent protein (GFP) and a polypeptide antagonist of a Na/K ATPase/Src receptor complex (nucleotide position 4626-4688; NaKtide; SEQ ID NO: 1) operably connected to a SGLT2 promoter (nucleotide position 1959-4595).

SEQ ID NO: 10 is a nucleic acid sequence of a lentivirus gene expression vector encoding a green fluorescent protein (GFP) and a polypeptide antagonist of a Na/K ATPase/Src receptor complex (nucleotide position 8060-8122; NaKtide; SEQ ID NO: 1) operably connected to a MyoD promoter (nucleotide position 1959-8029).

SEQ ID NO: 11 is a nucleic acid sequence of a lentivirus gene expression vector encoding a green fluorescent protein (GFP) and a polypeptide antagonist of a Na/K ATPase/Src receptor complex (nucleotide position 4167-4268, pNaKtide; SEQ ID NO: 5) operably connected to a glial fibrillary acidic protein (GFAP) promoter (nucleotide position 1959-4136).

SEQ ID NO: 12 is a nucleic acid sequence of a lentivirus gene expression vector encoding a green fluorescent protein (GFP) and a polypeptide antagonist of a Na/K ATPase/Src receptor complex (nucleotide position 2458-2559, pNaKtide; SEQ ID NO: 5) operably connected to a synapsin 1 (SYN1) promoter (nucleotide position 1959-2427).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

Additionally, while the terms used herein are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs. Although many methods, devices, and materials similar or equivalent to those described herein can be used in the practice or testing of the presently-disclosed subject matter, representative methods, devices, and materials are now described.

Furthermore, following long-standing patent law convention, the terms “a”, “an”, and “the” refer to “one or more” when used in this application, including the claims. Thus, for example, reference to “a polypeptide” includes a plurality of such polypeptides, and so forth. Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations in some embodiments of ±20%, in some embodiments of ±10%, in some embodiments of ±5%, in some embodiments of ±1%, in some embodiments of ±0.5%, and in some embodiments of ±0.1% from the specified amount, as such variations are appropriate to perform the disclosed method. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes expression vectors and related methods for targeted delivery of Na/K ATPase/Src receptor complex antagonists to specific cells and tissues, as well as methods for using such vectors to treat a Src-associated disease.

In some embodiments of the presently-disclosed subject matter, an expression vector is provided that includes a nucleic acid sequence encoding a polypeptide antagonist of a Na/K ATPase/Src receptor complex. The term “vector” is used herein to refer to any vehicle that is capable of transferring a nucleic acid sequence into another cell. For example, vectors which can be used in accordance with the presently-disclosed subject matter include, but are not limited to, plasmids, cosmids, bacteriophages, or viruses, which can be transformed by the introduction of a nucleic acid sequence of the presently-disclosed subject matter. In some embodiments, the vectors of the presently-disclosed subject matter are viral vectors, such as, in some embodiments, lentiviral vectors.

In some embodiments, the nucleic acid sequence included in the vector is operably linked to an expression cassette. The terms “associated with,” “operably linked,” and “operatively linked” refer to two nucleic acid sequences that are related physically or functionally. For example, a promoter or regulatory DNA sequence is said to be “associated with” or “operably linked” with a DNA sequence that encodes an RNA or a polypeptide if the two sequences are situated such that the regulator DNA sequence will affect the expression level of the coding or structural DNA sequence.

The term “expression cassette” or “expression vector” thus refers to a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression.

In some embodiments, an expression cassette is provided that comprises a promoter for directing expression of a nucleic acid sequence of the presently-disclosed subject matter in a particular cell or tissue. For example, in some embodiments, an adiponectin promoter is included in an expression cassette for directing expression of a particular nucleic acid of interest in adipose cells or tissue (see, e.g., SEQ ID NO: 6, which includes a nucleic acid sequence of a lentivirus gene expression vector including a polypeptide antagonist of the presently-disclosed subject matter operably connected to an adiponectin promoter). In some embodiments, the promoter can be an albumin promoter for directing expression of a nucleic acid sequence in hepatocytes (see, e.g., SEQ ID NO: 7, which includes a nucleic acid sequence of a lentivirus gene expression vector including a polypeptide antagonist of the presently-disclosed subject matter connected to an albumin promoter). In some embodiments, the promoter can be an alpha myosin heavy chain (αMHC) promoter for directing expression of a nucleic acid sequence in cardiomyocytes (see, e.g., SEQ ID NO: 8, which includes a nucleic acid sequence of a lentivirus gene expression vector including a polypeptide antagonist of the presently-disclosed subject matter connected to an αMHC promoter). In some embodiments, the promoter can be an SGLT2 promoter for directing expression of a nucleic acid sequence in the proximal tubule of a kidney (see, e.g., SEQ ID NO: 9, which includes a nucleic acid sequence of a lentivirus gene expression vector including a polypeptide antagonist of the presently-disclosed subject matter connected to a SGLT2 promoter). In some embodiments, the promoter can be a MyoD promoter for directing expression of a nucleic acid sequence in skeletal muscle (see, e.g., SEQ ID NO: 10, which includes a nucleic acid sequence of a lentivirus gene expression vector including a polypeptide antagonist of the presently-disclosed subject matter connected to a MyoD promoter). In some embodiments, the promoter can be a Glial Fibrillary Acidic Protein (GFAP) promoter for directing expression of a nucleic acid sequence in the brain including in astrocytes (see, e.g., SEQ ID NO: 11, which includes a nucleic acid sequence of a lentivirus gene expression vector including a polypeptide antagonist of the presently-disclosed subject matter connected to a GFAP promoter). In some embodiments, the promoter can be an Synapsin 1 (SYN1) promoter for directing expression of a nucleic acid sequence in brain tissue including mature neurons (see, e.g., SEQ ID NO: 12, which includes a nucleic acid sequence of a lentivirus gene expression vector including a polypeptide antagonist of the presently-disclosed subject matter connected to a SYN1 promoter). In some other embodiments, the promoter can be a melanin promoter for directing expressing of a nucleic acid sequence in melanoma tissue, or a von Willebrand factor promoter for directing expression of a nucleic acid sequence in endothelial cells. Of course, numerous other promoters known to those skilled in the art can also be chosen and utilized to direct expression of a nucleic acid sequence in a particular cell or tissue without departing from the spirit and scope of the subject matter described herein.

With respect to the nucleic acid sequences included in the expression vectors described herein, in some embodiments, the expression vectors include a nucleic acid sequence encoding a polypeptide of the sequence of SEQ ID NO: 1 (referred to herein as “NaKtide”), SEQ ID NO: 5 (referred to herein as “pNaKtide”), or fragments and/or variants thereof. In some embodiments, the polypeptides are comprised of the sequence of SEQ ID NO: 1 (NaKtide), or fragments, and/or variants thereof.

The terms “polypeptide,” “protein,” and “peptide” are used interchangeably herein to refer to a polymer of the protein amino acids regardless of its size or function. The terms “protein,” “polypeptide,” and “peptide” are used interchangeably herein to also refer to a gene product, homologs, orthologs, paralogs, fragments, any protease derived peptide (fragment), and other equivalents, variants, and analogs of a polymer of amino acids. The terms “polypeptide fragment” or “fragment” when used in reference to such a reference polypeptide, refer to a polypeptide in which amino acid residues are deleted as compared to the reference polypeptide itself, but where the remaining amino acid sequence is usually identical to the corresponding positions in the reference polypeptide. Such deletions may occur at the amino-terminus of the reference polypeptide, the carboxy-terminus of the reference polypeptide, or both. Polypeptide fragments can also be inclusive of “functional fragments,” in which case the fragment retains some or all of the activity of the reference polypeptide.

The term “variant,” as used herein, refers to an amino acid sequence that is different from the reference polypeptide by one or more amino acids. In some embodiments, a variant polypeptide may differ from a reference polypeptide by one or more amino acid substitutions. For example, a NaKtide polypeptide variant can differ from the NaKtide polypeptide of SEQ ID NO: 1 by one or more amino acid substitutions, i.e., mutations. In this regard, polypeptide variants comprising combinations of two or more mutations can respectively be referred to as double mutants, triple mutants, and so forth. It will be recognized that certain mutations can result in a notable change in function of a polypeptide, while other mutations will result in little to no notable change in function of the polypeptide.

In some embodiments, the present polypeptides include polypeptides that share at least 75% homology with the NaKtide polypeptide of SEQ ID NO: 1. In some embodiments, the polypeptides share at least 85% homology with the NaKtide polypeptide of SEQ ID NO: 1. In some embodiments, the polypeptides share at least 90% homology with the NaKtide polypeptide of SEQ ID NO: 1. In some embodiments, the polypeptides share at least 95% homology with the NaKtide polypeptide of SEQ ID NO: 1.

“Percent identity,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990).

In some embodiments of the presently-disclosed polypeptides, the polypeptides further comprise one or more leader sequences and, in some embodiments, leader sequences including, but not limited to, cell penetrating peptides (CPPs). The term “cell penetrating peptide” (CPP) is used herein to generally refer to short peptides that facilitate the transport of molecular cargo across plasma membranes found in a cell. In some instances, the molecular cargo includes another polypeptide, such as the polypeptides described herein. Of course, the cell penetrating peptides can be conjugated to the molecular cargo (e.g., polypeptide) via any number of means, including covalent bonds and/or non-covalent bonds. In a number of instances, however, such cell penetrating peptides will often include a relatively high concentration of positively-charged amino acids, such as lysine and arginine, and will have a sequence that contains an alternating pattern of charged (polar) and non-charged amino acids.

In some embodiments of the presently-disclosed subject matter, an exemplary leader sequence or cell-penetrating peptide can include the trans-activating transcriptional activator (TAT) cell penetrating peptide, which is represented by the sequence of SEQ ID NO: 2 (GRKKRRQRRRPPQ). Another exemplary leader sequence includes penetratin (AP), which is represented by the sequence of SEQ ID NO: 3 (RQIKIWFQNRRMKWKK). Yet another exemplary leader sequence includes an amino acid sequence encoding the N-terminal poly-lysine domain of the α1 subunit of Na/K-ATPase (A1N), which is represented by the sequence of SEQ ID NO: 4 (KKGKKGKK). Those of ordinary skill will appreciate though that other leader sequences, including other cell penetrating peptides, can also be used in conjunction with the presently-disclosed polypeptides. In some embodiments, a polypeptide including a leader sequence, such as a cell penetrating peptide, attached to the NaKtide sequence of SEQ ID NO: 1 is referred to herein as a pNaKtide (e.g., SEQ ID NO: 5; GRKKRRQRRRPPQSATWLALSRIAGLCNRAVFQ, which includes the TAT cell penetrating peptide of SEQ ID NO: 2 fused to the NaKtide sequence of SEQ ID NO: 1).

Further provided, in some embodiments of the presently-disclosed subject matter, are target cells transformed with the vectors disclosed herein. In some embodiments, the target cell is a mammalian cell, such as, in some embodiments, a mouse cell or a human cell. In some embodiments, the target cell is from a specific tissue such as, in some embodiments, an adipose cell, a liver cell, a melanoma cell, or an endothelial cell, among others.

The terms “transformed,” “transgenic,” and “recombinant” are used herein to refer to a cell of a host organism, such as a mammal, into which a heterologous nucleic acid molecule has been introduced. The nucleic acid molecule can be stably integrated into the genome of the cell or the nucleic acid molecule can also be present as an extrachromosomal molecule. Such an extrachromosomal molecule can be auto-replicating. Transformed cells, tissues, or subjects are understood to encompass not only the end product of a transformation process, but also transgenic progeny thereof.

The terms “heterologous,” “recombinant,” and “exogenous,” when used herein to refer to a nucleic acid sequence (e.g., a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides. Introduction of such nucleic acids (e.g., a nucleic acid incorporated into an appropriate vector) of the presently-disclosed subject matter into a plant cell can be performed by a variety of methods known to those of ordinary skill in the art

The presently-disclosed subject matter further includes and makes use of pharmaceutical compositions comprising the vectors described herein as well as a pharmaceutically-acceptable vehicle, carrier, or excipient. Indeed, when referring to certain embodiments herein, the terms “vector” and/or “composition” may or may not be used to refer to a pharmaceutical composition that includes the vector. In some embodiments, the pharmaceutical composition is pharmaceutically-acceptable in humans. Also, as described further below, in some embodiments, the pharmaceutical composition can be formulated as a therapeutic composition for delivery to a subject.

A pharmaceutical composition as described herein preferably comprises a composition that includes a pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of capsules, tablets or lozenges formulated in conventional manner.

Various liquid and powder formulations can also be prepared by conventional methods for inhalation into the lungs of the subject to be treated or for intranasal administration into the nose and sinus cavities of a subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently-disclosed subject matter and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

In addition to the formulations described above, the compositions of the presently-disclosed subject matter can also be formulated as rectal compositions, such as suppositories or retention enemas, e.g., containing conventional suppository bases such as cocoa butter or other glycerides. Further, the compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

As noted above, the presently-disclosed subject matter includes using vectors with specific promoters for delivery of an antagonist of a Na/K ATPase/Src receptor complex (e.g., a polypeptide of SEQ ID NO: 1 (NaKtide) or SEQ ID NO: 5 (pNaKtide)). In some embodiments, the vector targets the expression of pNaKtide or NaKtide to specific tissues, and thus avoids off target effects of the NaKtide or pNaKtide. In this regard, and still further provided by the presently-disclosed subject matter, are methods for treating a Src-associated disease. In some embodiments, a method for treating a Src-associated disease comprises administering an expression vector described herein to a subject in need thereof.

As used herein, the terms “treatment” or “treating” relate to any treatment of a condition of interest (e.g., a cancer), including, but not limited, to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest in a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

In some embodiments, the Src-associated disease is selected from the group consisting of cancer, vascular disease, cardiovascular disease, tissue fibrosis, and osteoporosis. In some embodiments, the Src-associated disease is selected from the group consisting of vascular disease, cardiovascular disease, heart disease, prostate cancer, breast cancer, neuroblastoma, cardiac hypertrophy, tissue fibrosis, congestive heart failure, ischemia/reperfusion injury, osteoporosis, retinopathy, and obesity.

In some embodiments, the Src-associated disease is cancer. In some embodiments, treating a cancer can include, but is not limited to, killing cancer cells, inhibiting the development of cancer cells, inducing apoptosis in cancer cells, reducing the growth rate of cancer cells, reducing the incidence or number of metastases, reducing tumor size, inhibiting tumor growth, reducing the available blood supply to a tumor or cancer cells, promoting an immune response against a tumor or cancer cells, reducing or inhibiting the initiation or progression of a cancer, or increasing the lifespan of a subject with a cancer.

As used herein, the term “cancer” refers to all types of cancer or neoplasm or malignant tumors found in animals, including leukemias, carcinomas, melanoma, and sarcomas. By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is selected from the group consisting of prostate cancer, breast cancer, and neuroblastoma.

In some embodiments, the Src-associated disease is cardiovascular disease, including, in some embodiments, uremic cardiomyopathy. In some embodiments, treating a cardiovascular disease can include, but is not limited to, reducing oxidative stress, reducing an amount of inflammatory cytokines, reducing cardiac fibrosis, and/or attenuating the development of diastolic dysfunction, cardiac hypertrophy, plasma creatinine levels, and anemia.

In some embodiments, the Src-associated disease is obesity. In some embodiments, treating obesity includes, but is not limited to, reducing an amount of subcutaneous and/or visceral fat, reducing an amount of body weight, reducing an amount of inflammatory cytokines, increasing an amount of oxygen consumption and/or energy expenditure, decreasing an amount of leptin, and reducing an amount of adipocity.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate kg factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments of the therapeutic methods described herein, the therapeutic compositions are administered orally, intravenously, intranasally, or intraperitoneally to thereby treat a disease or disorder.

Regardless of the route of administration, the compositions of the presently-disclosed subject matter typically not only include an effective amount of a therapeutic agent, but are typically administered in amount effective to achieve the desired response. As such, the term “effective amount” is used herein to refer to an amount of the therapeutic composition (e.g., a vector and a pharmaceutically vehicle, carrier, or excipient) sufficient to produce a measurable biological response (e.g., an increase in Src inhibition). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The examples may include compilations of data that are representative of data gathered at various times during the course of development and experimentation related to the presently-disclosed subject matter.

### EXAMPLES

### Example 1: In Vitro Transduction of Lentivirus with Adiponectin Promoter

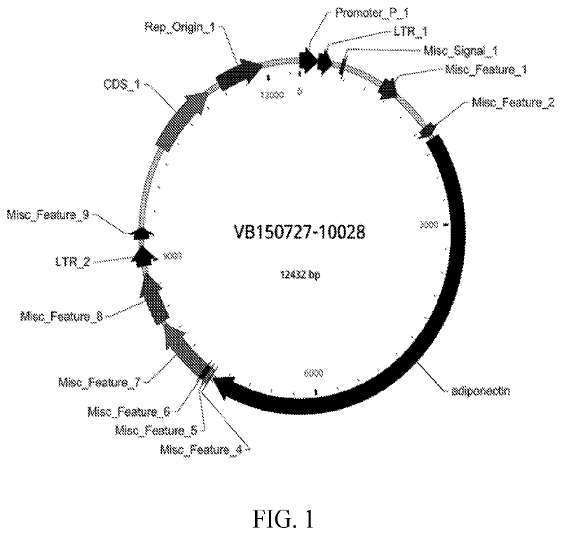

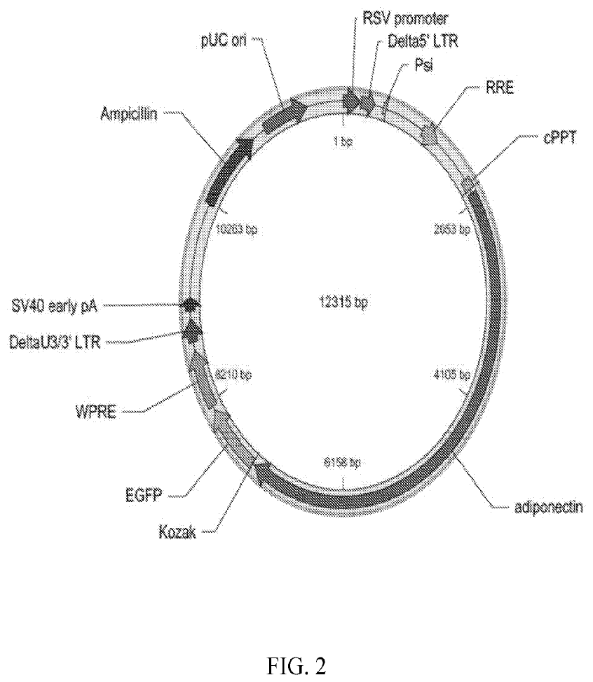

To target the expression of the NaKtide to adipose tissue, lentiviral vectors expressing either eGFP or eGFP-NaKtide cDNA under the control of an adiponectin promoter were constructed to achieve NaKtide expression specifically in adipocytes. 3T3-L1 preadipocytes (ATCC, VA) were used to evaluate functional transgene expression. Cells were then infected with the lentiviral vector (2 μl of 10⁹ TU/ml) carrying either the GFP-NaKtide (FIG. 1; SEQ ID NO: 6) or GFP (FIG. 2) construct under the control of the adiponectin promoter (Cyagen Biosciences, CA). A concentration curve was performed by infecting cells with 50, 100, or 200 MOI (multiplicity of infection). The effect of lentivirus-adiponectin-eGFP-NaKtide transduction in 3T3-L1 cells on lipogenesis, was evaluated with Oil Red O staining. GFP expression was confirmed using a confocal laser-scanning (Olympus Fluoview FV300) microscope and immunofluorescence was performed to detect NaKtide expression.

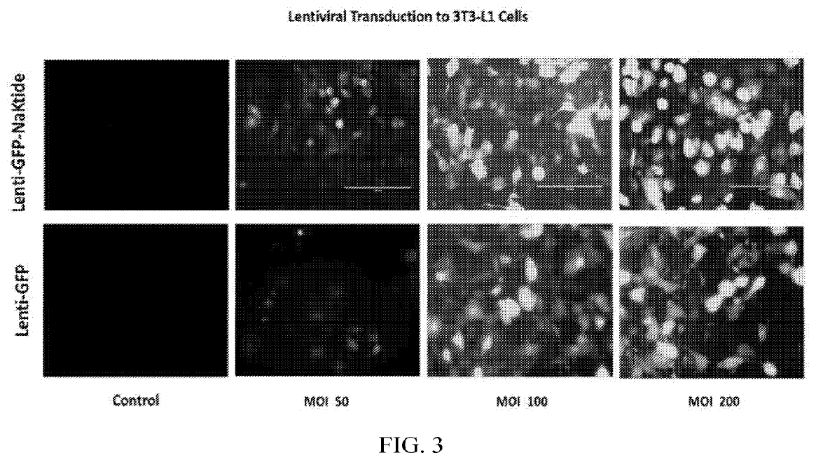

As shown in FIG. 3, fluorescent microscopy showed readily detectable GFP expression in both lenti-GFP and lenti-GFP-NaKtide adipocytes, as GFP fluorescence was evident in both groups, thus demonstrating the effectiveness of lentivirus-adiponectin-eGFP transduction in 3T3-L1 cells. Furthermore, the increasing MOI in both groups demonstrated increasing GFP fluorescence, indicating there was an increase in transduced cells with increased MOI.

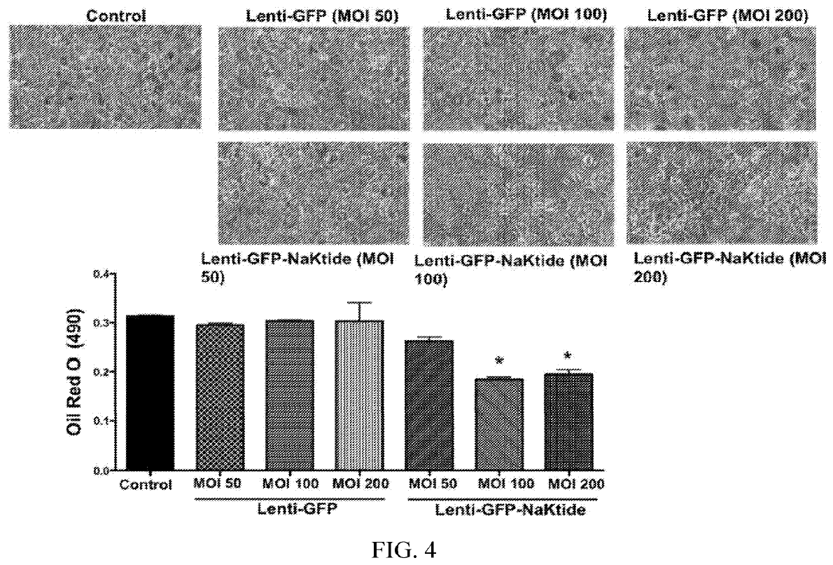

3T3-L1 cells infected with increasing MOI of Lenti-Adiponectin-eGFP-NaKtide or Lenti-Adiponectin-eGFP were also stained with oil red O after 7 days, which stains for lipids, to determine whether NaKtide expression had an effect on lipogenesis (FIG. 4). Infection with 100 and 200 MOI of lenti-adiponectin-eGFP-NaKtide significantly decreased (p<0.05) oil red O staining compared to control and MOI 50. There was however, no difference between infecting with 100 and 200 MOI. Transduction with Lenti-Adiponectin-eGFP showed no effect on lipogenesis compared to control cells, regardless of MOI.

### Example 2: Lentiviral-Mediated Delivery of NaKtide in C57BL/6 Mice with Adiponectin Promoter

To assess the in vivo introduction of a lentiviral construct driven by an adiponectin promoter, and the resulting expression of NaKtide specifically in adipose tissue, C57BL/6 male mice (4-6 weeks) were used. The lentiviral constructs with mouse NaKtide, driven by an adiponectin promoter (FIGS. 1 and 2) were used in mice to achieve NaKtide expression specifically in adipose tissues. Lentivirus (100 μl, 2×10⁹ TU/ml in saline) with NaKtide, and its counterpart Lenti-eGFP, driven by an adiponectin promoter, were injected into mice by intra peritoneal injection. Two weeks later, another intra peritoneal injection (75 μl 1×10⁹ TU/ml) was given.

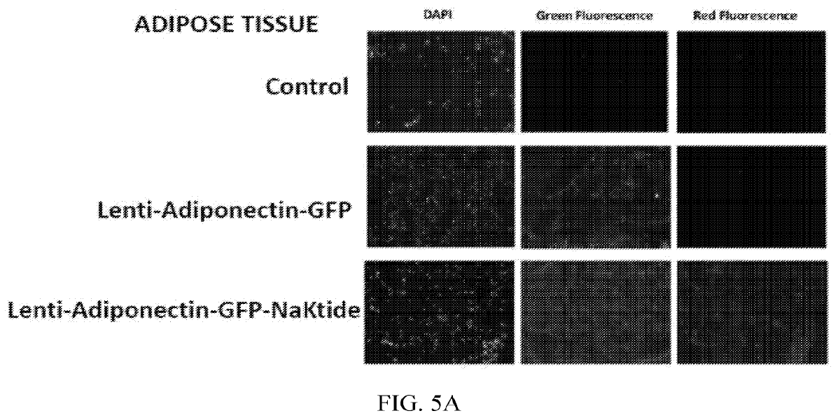

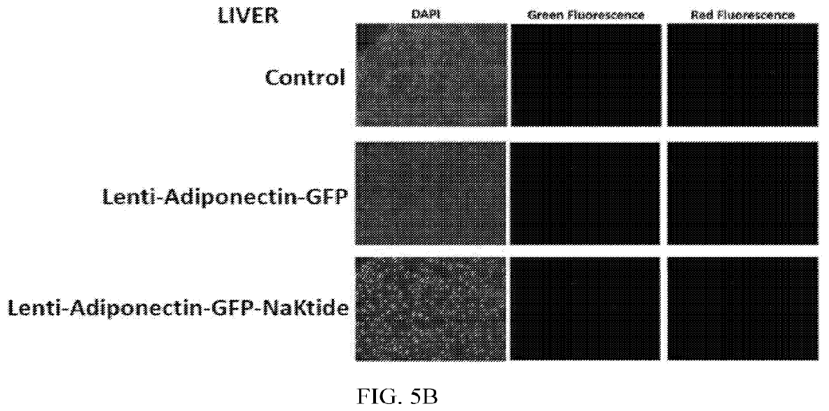

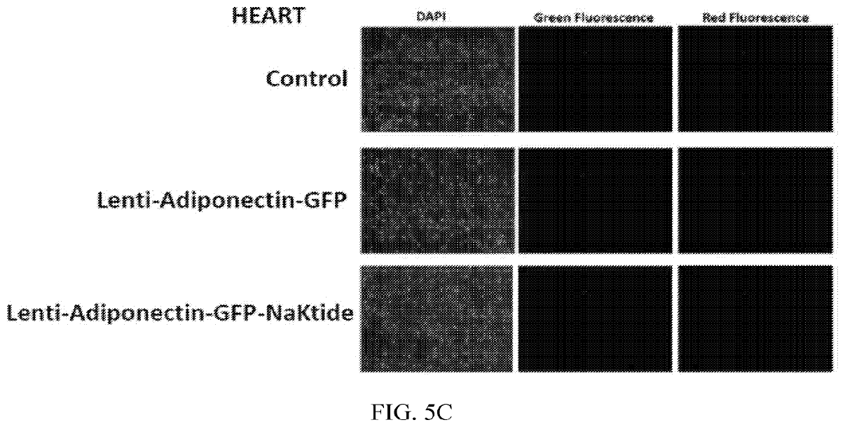

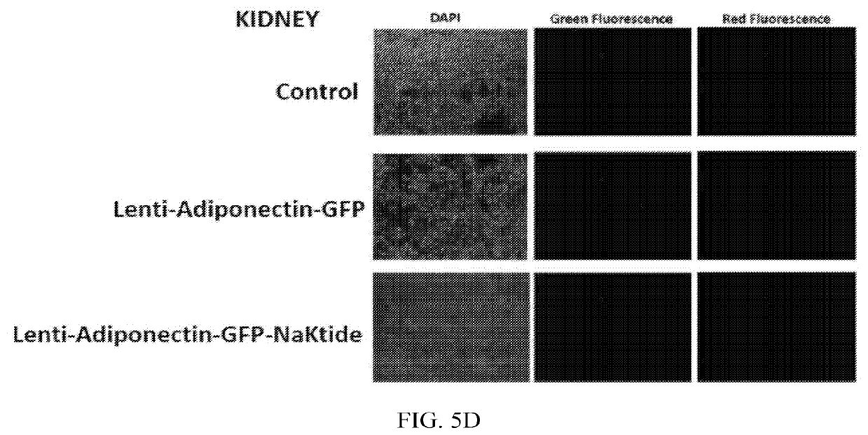

Immunofluorescence was used to investigate the effectiveness of lentivirus-adiponectin-eGFP gene targeting in the C57BL/6 mice. Adipose, liver, and heart tissues were harvested from mice injected with lenti-adiponectin-eGFP and Lenti-adiponectin-eGFP-NaKtide. Fluorescent microscopy showed readily detectable GFP expression in both adipose sections (lenti-adiponectin-eGFP and lenti-adiponectin-eGFP-NaKtide) (FIG. 5A) and no detectable expression in liver (FIG. 5B), heart (FIG. 5C), and kidney (FIG. 5D) tissues, indicating that the adiponectin promoter was effective in driving expression of the lentivirus, selectively in adipose tissues. Immunofluorescence was also performed using a NaKtide primary polyclonal antibody and Alexa Fluor 555 polyclonal secondary antibody on all tissue sections. This immunofluorescence staining demonstrated that NaKtide was detected only in the adipose tissues of lenti-adiponectin-eGFP-NaKtide injected mice (FIG. 5A). Overexpression of the NaKtide gene only in adipose tissue of lenti-adiponectin-NaKtide mice showed the effectiveness and specificity of the lenti-adiponectin-NaKtide promoter in these mice.

### Example 3: Lentiviral-Mediated Delivery of the NaKtide in Live Animals

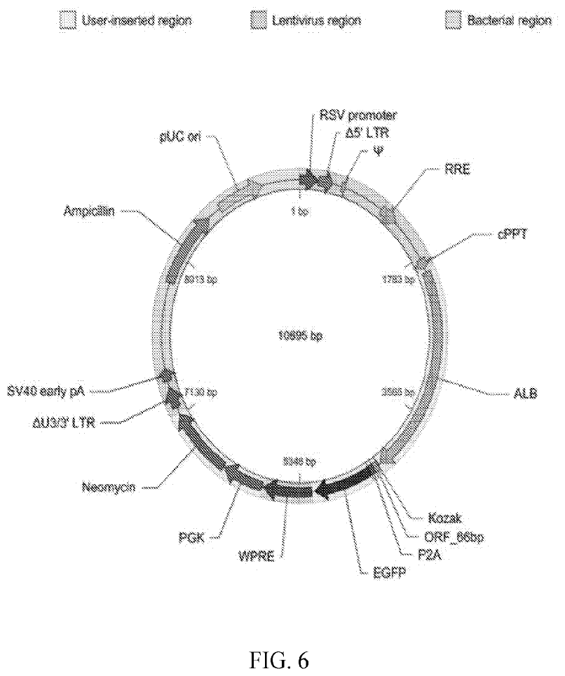

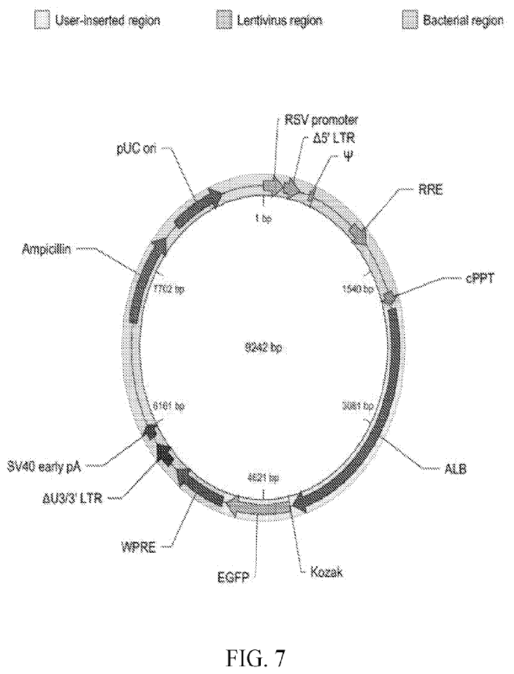

To assess lentiviral-mediated delivery of the NaKtide in live animals, C57BL/6 male mice (4-6 weeks) were again used. A lentiviral construct with mouse NaKtide, driven by an albumin promoter, was constructed to achieve NaKtide expression specifically in the liver. This mode of intervention was utilized to obtain NaKtide expression for an extended period of time. Lentivirus (100 2×10⁹ TU/ml in saline) with eGFP-NaKtide (FIG. 6; SEQ ID NO: 7) and its counterpart Lenti-eGFP (FIG. 7), driven by an albumin promoter, were injected into mice by intra peritoneal injection. Two weeks later, another injection (75 μl 1×10⁹ TU/ml i.p.) was given.

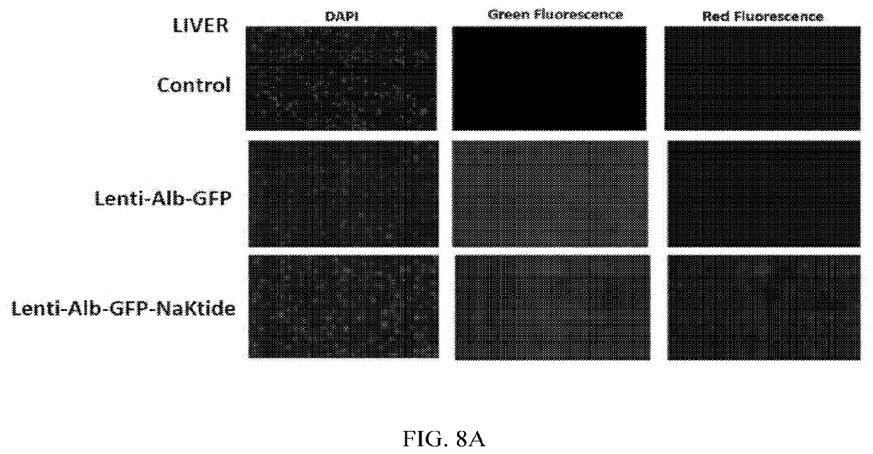

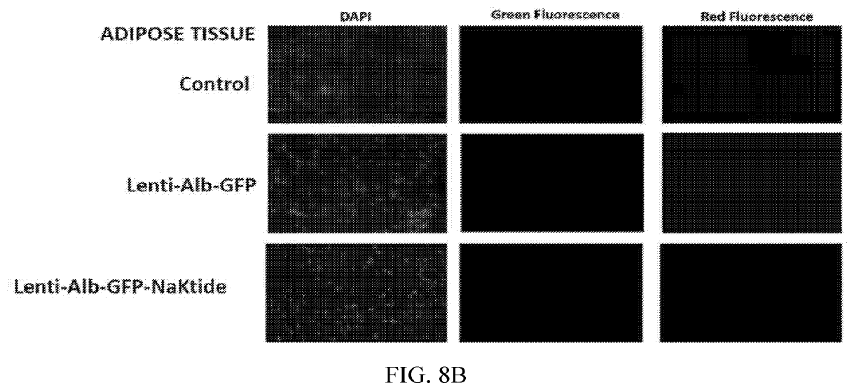

After harvesting the liver and adipose tissues from mice injected with Lenti-Alb-eGFP and Lenti-Alb-eGFP-NaKtide, fluorescent microscopy showed readily detectable GFP expression in both liver sections (Lenti-Alb-eGFP and Lenti-Alb-eGFP-NaKtide) and no detectable expression in adipose tissue, indicating that the albumin promoter was effective in driving expression of the lentivirus, selectively in hepatic tissues (FIGS. 8A and 8B). Immunohistochemistry (IHC) was also performed using a NaKtide primary monoclonal antibody and Alexa Fluor 555 polyclonal secondary antibody on liver and adipose tissue sections. This immunohistochemistry (IHC) staining demonstrated that NaKtide was detected only in the liver of Lenti-Alb-eGFP-NaKtide injected mice.

### Example 4—NaKtide Targeting to Adipocytes Attenuates Adiposity and Systemic Oxidative Stress in Mice Fed a Western Diet by Reprogramming Adipocyte Phenotype

To determine the effect of adipocyte-specific NaKtide expression on adiposity and systemic oxidative stress, animal studies were first approved by the Marshall University Animal Care and Use Committee in accordance with the National Institutes of Health (NIH) Guide for the Care and Use of Laboratory Animals. C57Bl6 mice (6 to 8 weeks old, male) were purchased from Hilltop Lab Animals. Upon arrival to the Robert C. Byrd Biotechnology Science Center Animal Resource Facility (ARF), the mice were placed in cages and fed normal chow and had access to water ad libitum. Western diet (WD) containing fructose is a well-known model of diet induced obesity. WD was purchased commercially from Envigo (Indianapolis, Ind.). WD contained 42% fat, 42.7% carbohydrate, and 15.2% protein yielding 4.5 KJ/g. Fructose was purchased commercially from Alfa Aesar (Ward Hill, Mass.). Fructose was made at a concentration of 42 g/L, yielding 0.168 KJ/mL. WD mice were given WD chow and had ad libitum access to high fructose water. The animals were randomly divided into five groups; 1) normal chow, 2) normal chow+lentiviral-GFP-NaKtide (SEQ ID NO: 6), 3) WD, 4) WD+lentiviral-GFP, and 5) WD+lentiviral-GFP-NaKtide (n=12 to 14 per group) and placed on their respective diets. The lentiviral constructs with mouse NaKtide, driven by an adiponectin promoter, were used in mice to achieve NaKtide expression specifically in adipose tissues. Lentivirus (100 μl, 2×10⁹ TU/ml in saline) with NaKtide, and its counterpart Lenti-eGFP, driven by an adiponectin promoter, were injected into mice intraperitoneally. Two weeks later, another injection (75 μl 1×10⁹ TU/ml i.p.) was given. Groups 2 and 5 were given an injection of lenti-adipo-NaKtide and group 4 was given an injection of lenti-adipo-GFP at Week 0 and again at week 2. Body weight was measured weekly, as well as food and water intake. At the time of sacrifice, the body weight and visceral and subcutaneous fat content of all mice were measured. Blood samples were collected for determination of inflammatory cytokine levels. Tissues were flash-frozen in liquid nitrogen and maintained at −80° C.

For the assessment of indirect calorimetry and locomotor activity, at the end of the 12-week experimental period, energy expenditure and locomotor activity were measured using an eight-chamber CLAM (Columbus Instruments, Columbus, Ohio, USA). In this system, total oxygen consumption (VO₂) and carbon dioxide production (VCO₂) were measured, and VO₂ was converted to individual heat production (kcal/hour) by Columbus software. This software calculates the heat production by multiplying the calorific value CV=3.815+(1.232×RER) by the observed VO₂ (Heat=CV×VO₂). The energy expenditure was then calculated as a ratio of heat produced divided by body mass. A system of infrared beams detects movement of animals in CLAMS, and locomotor activity was determined as ambulatory count, the number of times different beams were broken in either the x- or y-axes during an interval. All mice were acclimatized to monitoring cages for 24 hours prior to an additional 48 hours of recordings under the regular 12-hour light-dark cycle.

For the glucose tolerance test, glucose clearance was determined using an intraperitoneal glucose tolerance test before termination of the experiment. Mice were fasted for 8 hours, after which a glucose solution (2 g/kg, injected as a 10% solution) was injected into the peritoneal cavity. Samples were taken from the tail vein at 0, 30, 60, and 120 min after glucose injection. Blood glucose was measured using the Accutrend Sensor glucometer.

For cytokine measurements, IL-6, MCP-1, and TNFα cytokine measurements were performed using an ELISA assay kit according to manufacturer instructions (Abcam).

For the measurement of c-Src phosphorylation, whole cell lysates from visceral adipose tissue were prepared with RIPA buffer and activation of c-Src was determined as previously described. After immunoblotting for phospho-c-Src, the same membrane was stripped and immunoblotted for total c-Src. Activation of c-Src was expressed as the ratios of phospho-c-Src/total Src with measurements normalized to 1.0 for the control samples.

For the assessment of protein carbonylation, whole-cell lysates from visceral adipose tissues were prepared with RIPA buffer and western blotting for protein carbonylation assay was done. The signal density values of control samples were normalized to 1.0 with Coomassie blue staining as a loading control.

For western blot analysis, visceral adipose tissue was pulverized with liquid nitrogen and placed in a homogenization buffer. Homogenates were centrifuged, the supernatant was isolated, and immunoblotting was performed. The supernatant was used for the determination of FAS, PPARy, MEST, and PGC1α as previously reported. Loading conditions were controlled for using GAPDH.

For haematoxylin and eosin staining, the aorta, stored in OCT, was cut into 6 μm sections and stained with haematoxylin and eosin for histological analysis.

In the above-described methods, statistical significance between experimental groups was determined by the Tukey post hoc method of analysis of multiple comparisons (P<0.05). For comparisons among treatment groups, the null hypothesis was tested by a one way analysis of variance (ANOVA). Data are presented as means±SE.

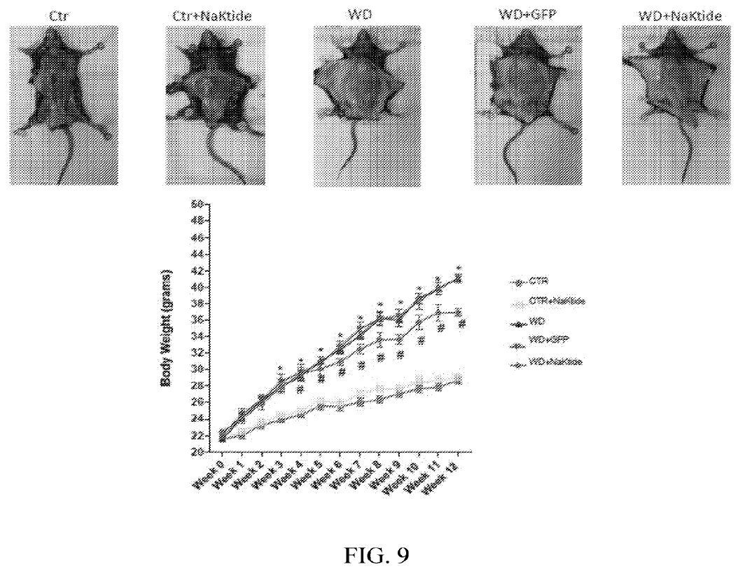

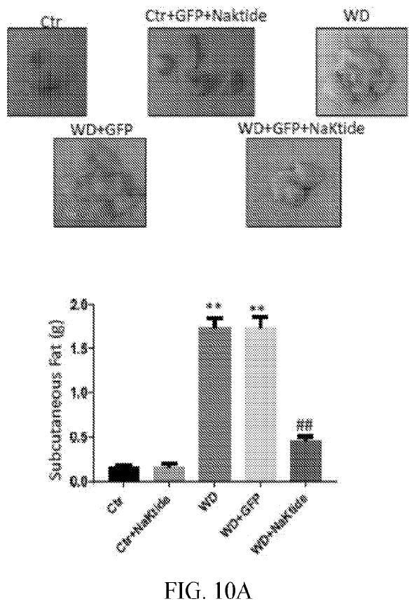

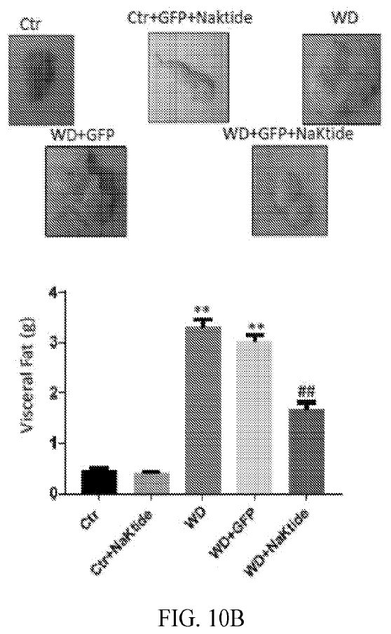

Upon obtaining the results of the experiments, the effect of adipocyte-specific NaKtide expression on body weight, and visceral and subcutaneous fat content in mice fed a western diet was first examined. Mice fed a western diet exhibited an increase in body weight over a period of 12 weeks compared to the mice on normal chow diet. Mice transduced with adiponectin-NaKtide showed a significant decrease in weight gain over the course of the 12 week period as compared to mice fed a western diet (FIG. 9). Groups treated with GFP alone showed no difference compared to the respective control groups. Mice receiving adiponectin-NaKtide and fed a western diet also showed marked reduction in both subcutaneous and visceral fat as compared to mice fed a western diet (FIGS. 10A-10B). These observations supported the hypothesis that adipocyte-specific targeted NaKtide using a lentivirus construct can attenuate adiposity.

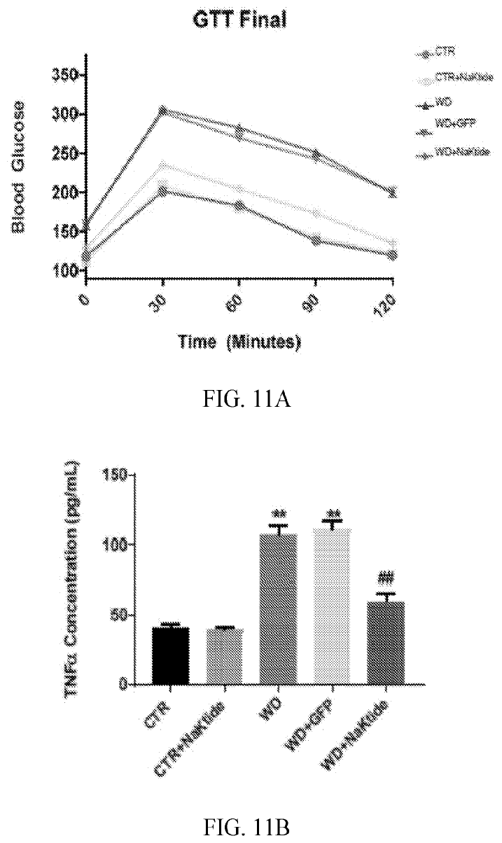

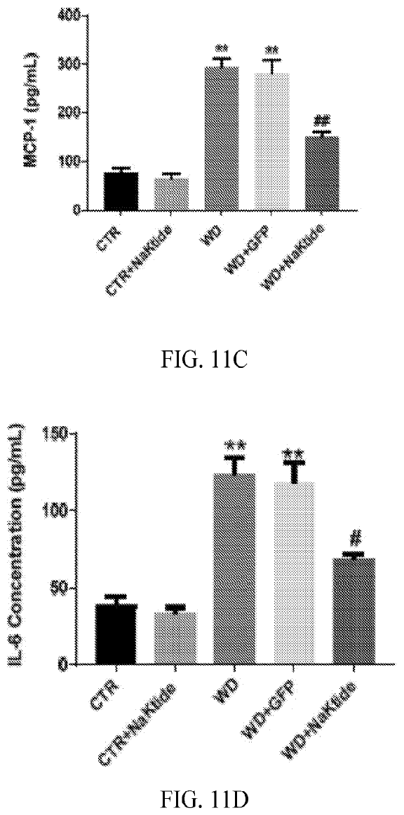

Next, the effect of adipocyte-specific NaKtide expression on glucose tolerance test and inflammatory cytokines in mice fed a western diet was examined. Mice fed a western diet exhibited a decreased glucose tolerance compared to the mice on normal chow diet. Mice receiving lenti-adiponectin-NaKtide fed a western diet showed an improved glucose tolerance compared to mice fed a western diet (FIG. 11A). Groups treated with GFP alone showed no difference compared to the respective control groups.

Mice fed a western diet showed higher levels of these cytokines compared to control groups. Lenti-adiponectin-NaKtide administration in mice fed western diet showed significantly lower levels of the inflammatory cytokines TNFα and MCP-1 compared to mice fed a western diet (FIGS. 11B-11D). Groups treated with GFP alone showed no difference compared to the respective control groups.

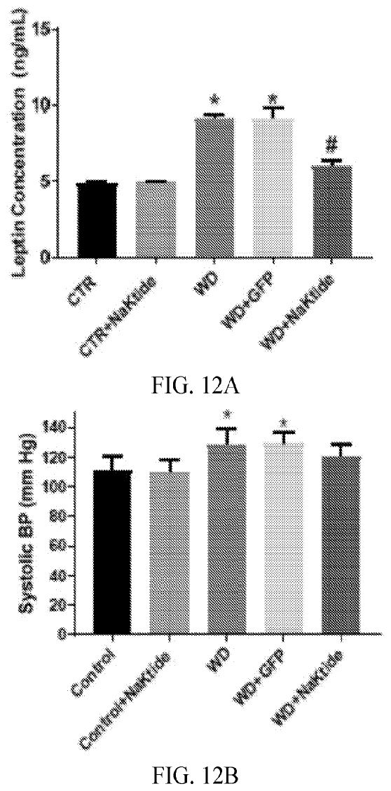

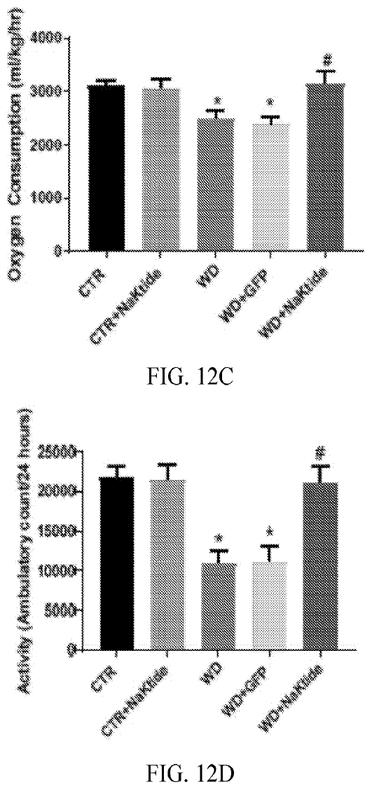

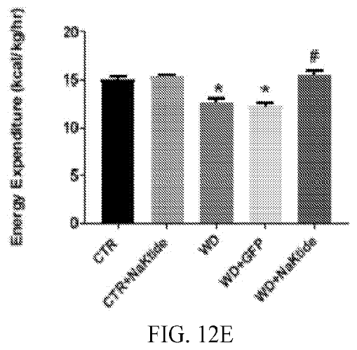

The effect of adipocyte-specific NaKtide expression on leptin, systolic blood pressure, oxygen consumption, activity, and energy expenditure in mice fed a western diet was also analyzed. Mice fed a western diet exhibited significantly increased plasma leptin concentrations compared to the mice on a normal chow diet; this was ameliorated in lenti-adiponectin-NaKtide treated mice (FIG. 12A). The systolic blood pressure of western diet mice was also significantly higher than those of their control counterparts, and the WD NaKtide treated mice (FIG. 12B).

When placed in CLAMS cages it was found that mice fed a western diet showed lowered oxygen consumption, activity, and energy expenditure compared to the control groups. Mice receiving lenti-adiponectin-NaKtide had increases in oxygen consumption, activity, and energy expenditure compared to western diet alone (FIGS. 12C-12E).

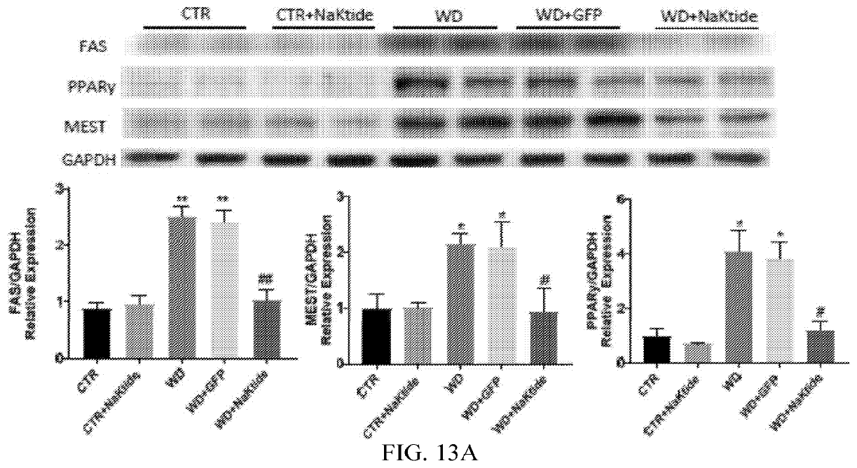

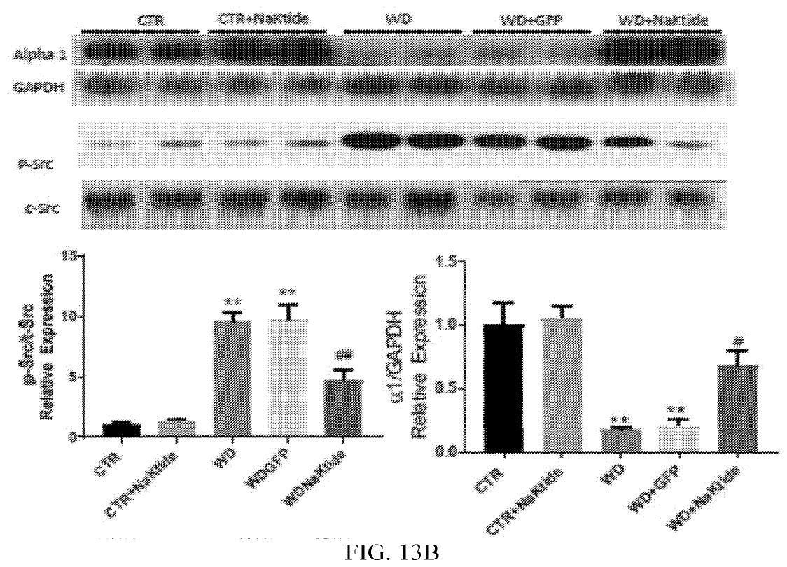

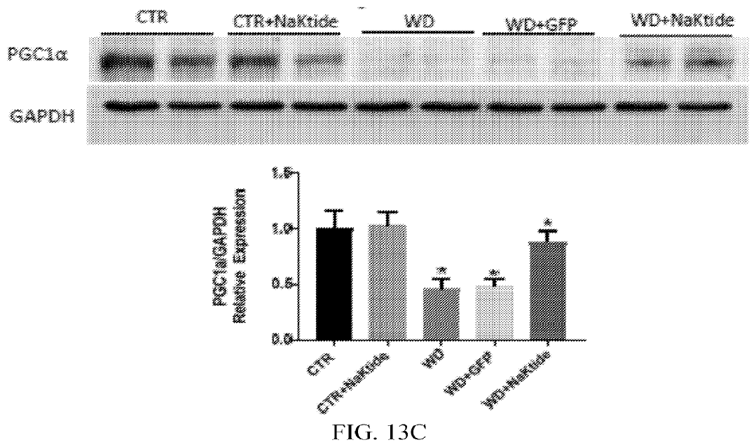

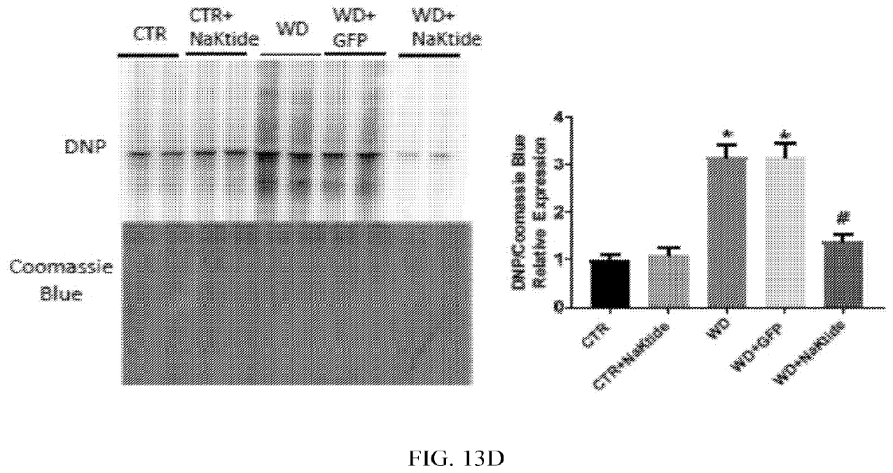

The effect of adipocyte specific NaKtide expression on adipogenesis related proteins, Na/K-ATPase signaling markers, and brown fat marker PGC1α in mice fed a western diet was further determined. Mice fed a western diet exhibited increased expression of FAS, PPARy, and MEST (FIG. 13A). Fatty acid synthase (FAS) and peroxisome proliferator-activated receptor gamma (PPARy) are involved in adipocyte growth and development, and mesoderm specific transcript (MEST) is a marker of adipocyte size. Lenti-adiponectin-NaKtide treated mice had lowered levels of protein expression compared to the western diet fed animals. Phosphorylation of Src (a downstream target of Na/K-ATPase signaling) was increased in mice fed a western diet, and decreased in mice treated with lenti-adiponectin-NaKtide (FIG. 13B). Expression of the alpha 1 subunit of the Na/K-ATPase was significantly decreased in western diet fed mice, and rescued in mice treated with lenti-adiponectin-NaKtide (FIG. 13B). Carbonylation of the alpha 1 subunit of the Na/K-ATPase (a marker of oxidative stress) was increased in mice fed with western diet, and decreased in lenti-adiponectin-NaKtide treated mice (FIG. 13D).

PGC1α is a protein associated mitochondrial biogenesis and thermogenic regulation. In visceral fat of mice fed with a western diet, PGC1α expression was significantly decreased. Treatment with lenti-adiponectin-NaKtide increases the expression of PGC1α compared to WD fed mice (FIG. 13C).

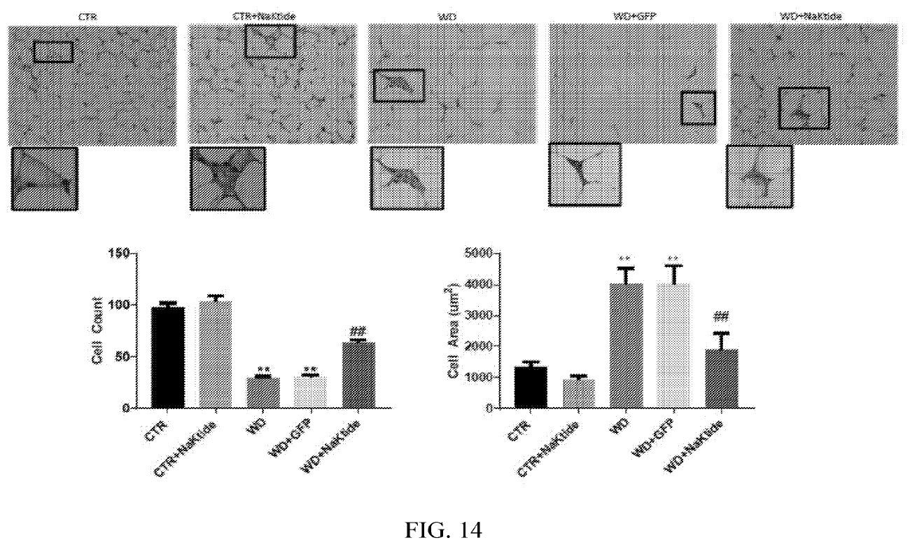

In examining the effect of adipocyte specific NaKtide expression on adipocyte size and number in visceral fat in mice fed a western diet, it was observed that mice fed a western diet showed significantly increased area of adipose tissue, with a significant reduction in cell number compared to control animals as shown through H&E staining. Treatment with lenti-adiponectin-NaKtide increased cell count and decreased the overall area of the cells (FIG. 14).

### Example 5—Role of Na/K-ATPase Signaling in Adipocytes in the Development and Progression of Uremic Cardiomyopathy in Murine PNx Model

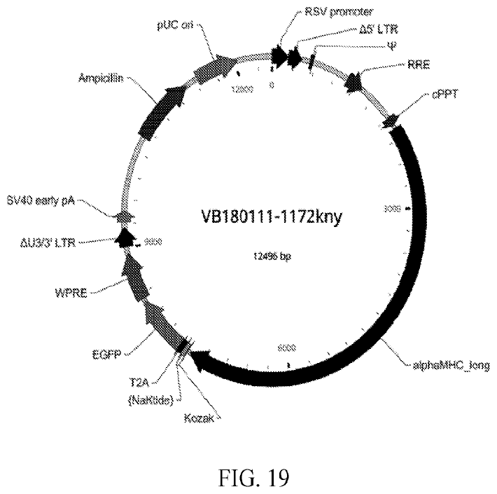

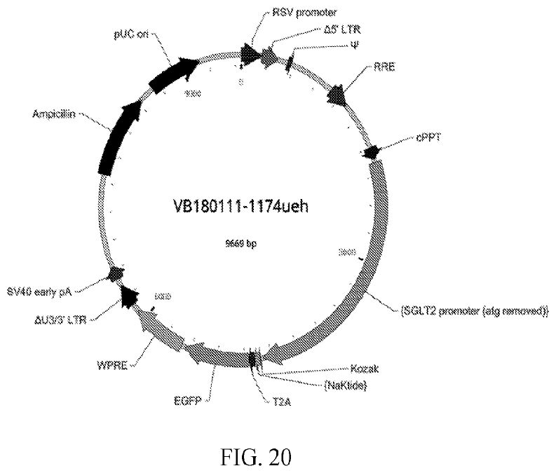

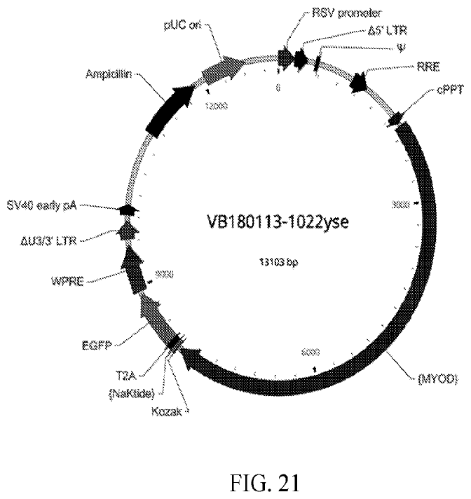

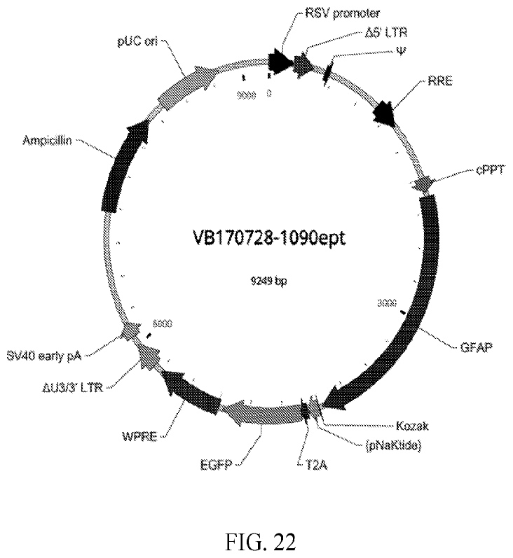

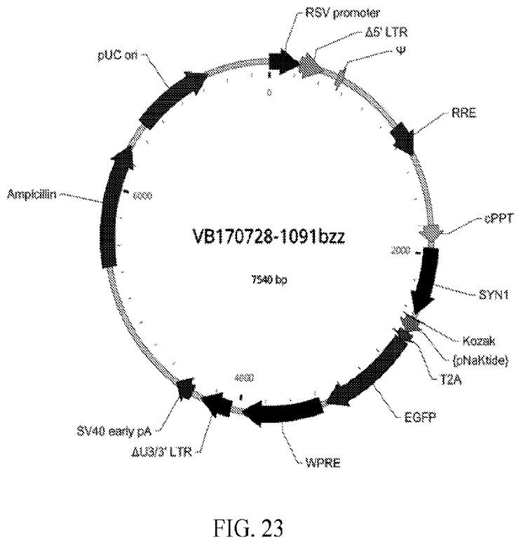

For the experiments undertaken to assess the role of Na/K-ATPase signaling in adipocytes in the development and progression of uremic cardiomyopathy, animal studies were approved by the Marshall University Animal Care and Use Committee in accordance with the National Institutes of Health (NIH) Guide for the Care and Use of Laboratory Animals. C57Bl6 mice (6 to 8 wks old, male) were purchased from Hilltop laboratories. Upon arrival to the Robert C. Byrd Biotechnology Science Center Animal Resource Facility (ARF), mice were placed on a normal chow diet containing 11% fat, 62% carbohydrate, and 27.0% protein with total calories of 12.6 KJ/g and had free access to water or the mice were placed on Western Diet (WD) containing 42% fat, 42.7% carbohydrate, and 15.2% protein yielding 4.5 KJ/g and had free access to high fructose solution (42 g/L), yielding 0.168 KJ/mL. To mimic uremic cardiomyopathy, 5/6-nephrectomy (PNx) mouse models, C57Bl6 male mice (10-12 weeks old) purchased from Jackson Laboratories were used. PNx surgeries were performed as described previously. Briefly the PNx model uses a two-step surgical approach. The first step is to surgically ligate the superior and inferior poles of the left kidney so only ½ of the left kidney mass is functional. The second step is to remove the right kidney 7 days post-ligation. For sham controls, the surgical steps are repeated without removing the kidneys. Lentiviral vectors containing eGFP and NaKtide (an antagonist of Na/K-ATPase/Src signaling pathway) or the respective control eGFP, was injected into the C57BL/6 mice using the LentiMax™ system for this study. The eGFP-NaKtide or eGFP control was under the control of an adiponectin, alpha-MHC, SGLT2 or MyoD specific promoter, to target adipocytes, cardiomyocytes, the apical side of the renal proximal tubal cell and skeletal muscle respectively (FIGS. 1 and 19-21, and SEQ ID NOS: 6, 8, 9, and 10, respectively). Lentivirus (100 μl, 2×10⁹ TU/ml in saline) was injected into the C57BL/6 mice i.p. Appropriate pre and post-surgical care was taken according to IACUC rules and regulations. Mice were weighed every week and blood pressure was determined by tail cuff method immediately prior to surgery and then every 4 weeks after surgery. At the time of sacrifice, the body weight and visceral and subcutaneous fat content of all mice were measured. Blood samples were collected for determination of inflammatory cytokine levels. Tissues were flash-frozen in liquid nitrogen and maintained at −80° C.

For a glucose tolerance test, glucose clearance was determined using an intraperitoneal glucose tolerance test before termination of the experiment. Mice were fasted for 8 hours, after which a glucose solution (2 g/kg, injected as a 10% solution) was injected into the peritoneal cavity. Samples were taken from the tail vein at 0, 30, 60, and 120 min after glucose injection. Blood glucose was measured using the Accutrend Sensor glucometer.

For cytokine measurements in these experiments, MCP-1 and TNFα cytokine measurements were performed using an ELISA assay kit according to manufacturer instructions (Abcam).

For TBARS Measurement, TBARS measurement was performed using TBARS Parameter Assay Kit (R&D Systems) according to manufacturer's protocol.

For RT-PCR, RNA Extraction was performed using miRNeasy SerumPlasma Kit (Qiagen, Hilden, Germany). The manufacturer's protocol was followed to extract RNA from serum samples and further analyze the quantity and quality of the RNA by 260:280 ratio using NanoDrop Analyzer (Thermo Scientific). Following the RNA extraction, miRCURY LNA Universal RT microRNA PCR Kit (Exiqon, Vedbaek, Denmark) was used for the RT reactions, to prepare cDNA, with 50 ng of total RNA for each reaction. Further, miRNA specific primers were used combined with SYBR green master mix to perform RT-PCR reaction. Three technical replicates were used for each sample allowing more accuracy in the final qRT-PCR amplification data which was run on a 7500 Fast Real Time PCR System (Applied Biosystems).

To assess cardiac function, systolic/diastolic blood pressure was measured in the mice using the CODA 8-Channel High Throughput Non-Invasive Blood Pressure system (Kent Scientific Corporation) that measures blood pressure in up to 8 mice simultaneously. Transthoracic echocardiography (TTE) was performed for the assessment of cardiac hypertrophy by measuring left ventricular mass, ejection fraction, myocardial performance index and relative wall thickness.

In the above-described experiments for this example, statistical significance between experimental groups was determined by the Tukey post hoc method of analysis of multiple comparisons (P<0.05). For comparisons among treatment groups, the null hypothesis was tested by a one way analysis of variance (ANOVA). Data are presented as means±SEM.

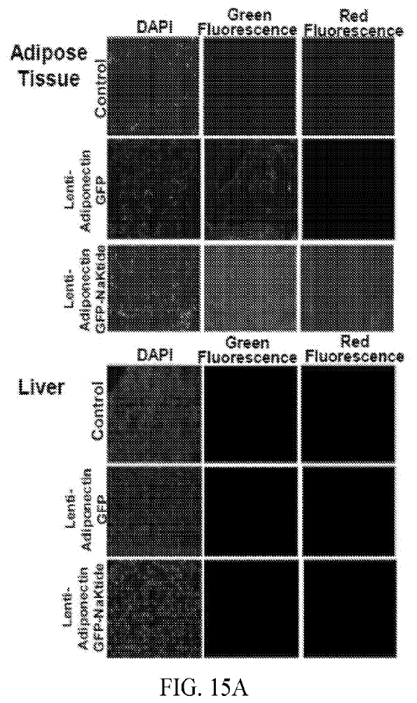

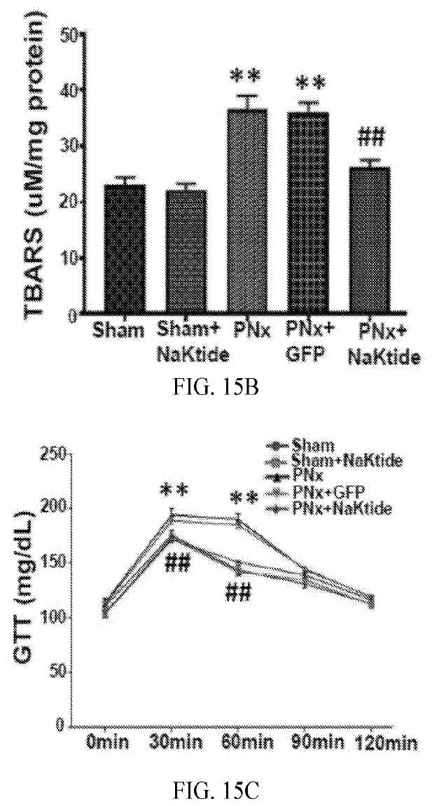

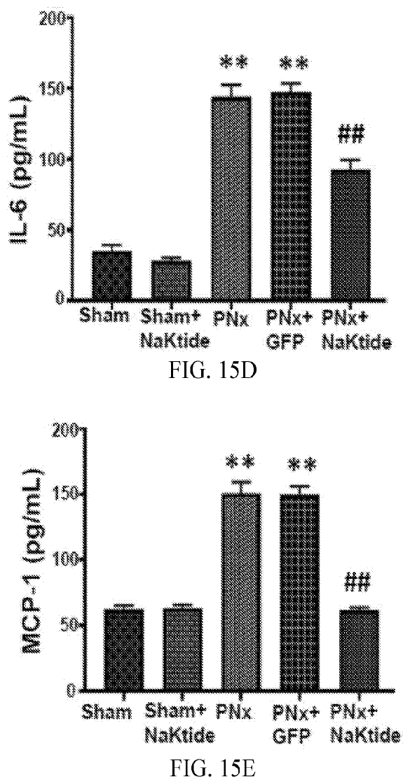

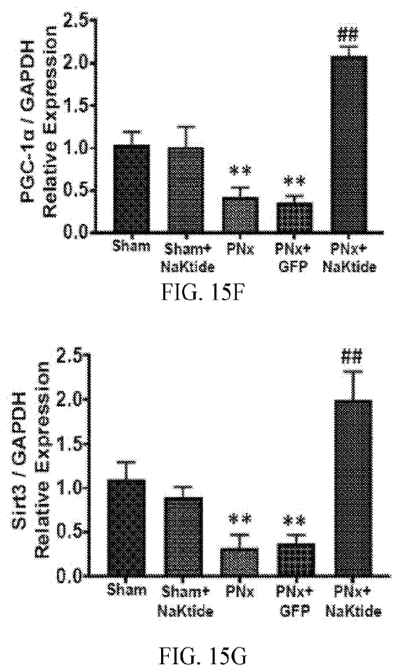

Upon analysis of the results, it was observed that lenti-adiponectin-NaKtide targeting specifically to adipocytes attenuates oxidative stress, improves metabolic profile, mitochondrial biogenesis and adaptive thermogenesis in a murine experimental uremic cardiomyopathy model. To assess the effectiveness and specificity of lentivirus gene targeting, adipose and liver tissues were harvested from C57BL/6 mice, injected with Lenti-adiponectin-eGFP and Lenti-adiponectin-eGFP-NaKtide (FIG. 15A). Immunofluorescence staining demonstrated readily detectable GFP and NaKtide expression in adipose sections, while no detectable expression was noted in liver tissues. In the study, mice were injected with Lenti-adiponectin-GFP-NaKtide as described above followed by partial nephrectomy (PNx) on the same day, to establish a model of experimental uremic cardiomyopathy. The results showed that Lenti-adiponectin-NaKtide ameliorated oxidative stress, glucose tolerance and significantly reduced cytokine levels in C57BL/6 PNx model (FIGS. 15B-15E). PGC-1α and Sirt3 are well-established markers that mediate mitochondrial biogenesis and causes browning of white fat (thermogenic fat). RT-PCR analyses showed that Lenti-adiponectin-NaKtide significantly improved PGC-la and Sirt3 expression, indicating improved mitochondrial biogenesis and restored thermogenic function (FIGS. 15F-15G).

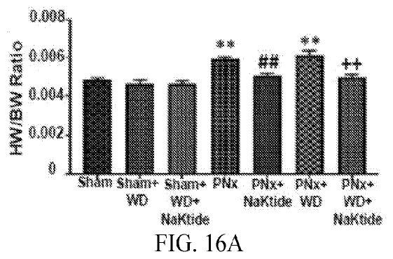

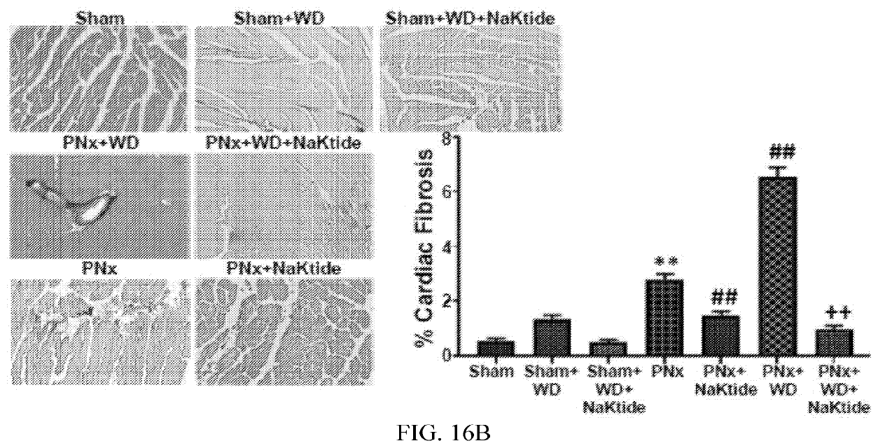

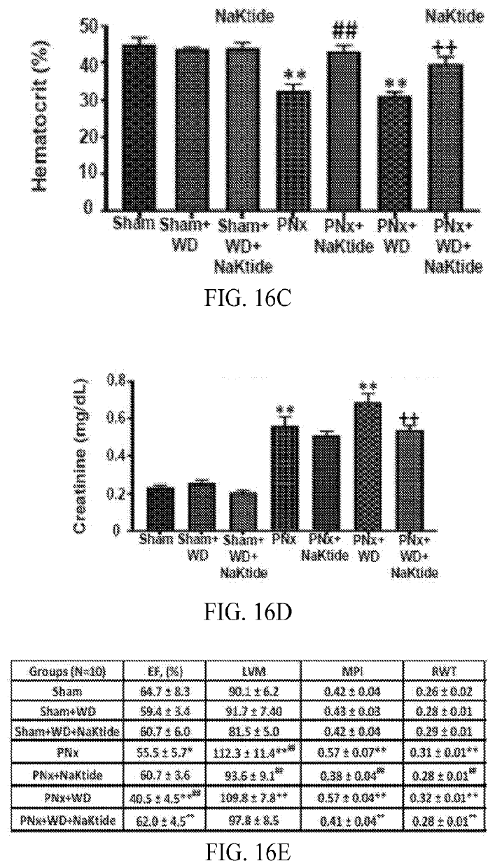

The ability of the lenti-adiponectin-NaKtide construct to target specifically to adipocytes and attenuate uremic cardiomyopathy was next assessed. In addition to the effects on cardiac fibrosis, NaKtide targeted specifically to adipocytes attenuated the development of diastolic dysfunction (assessed with Echo measurements), cardiac hypertrophy (assessed by heart weight/body weight ratio as well as LVMI, and wall thickness on Echo), plasma creatinine levels, and anemia seen with experimental renal failure in the mouse (FIGS. 16A-16E). BP effects of NaKtide were minimal, as the C57BL/6 PNx model does not produce significant hypertension.

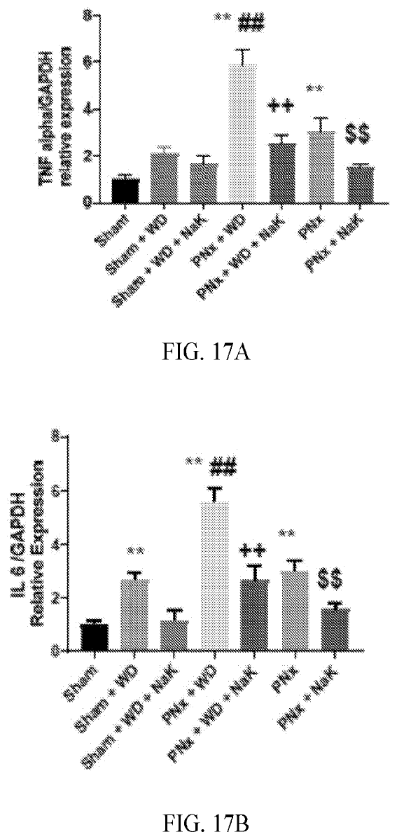

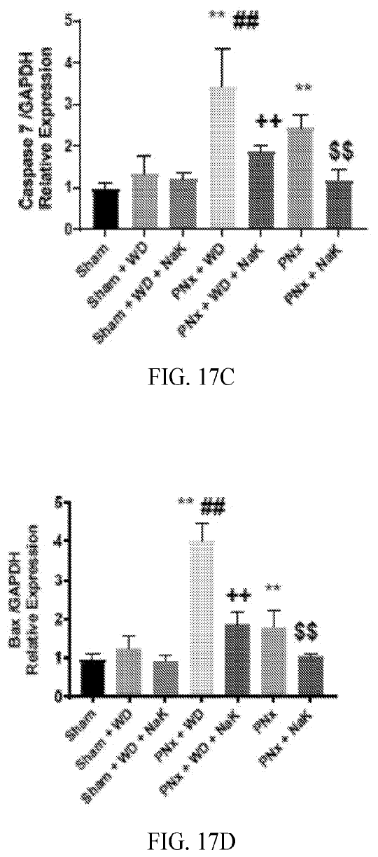

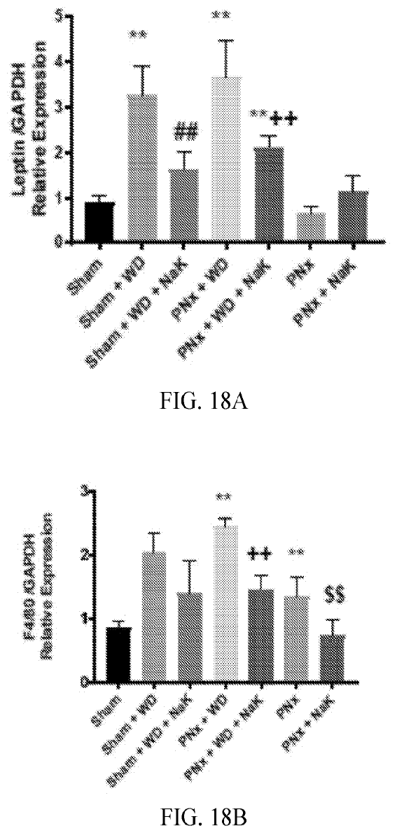

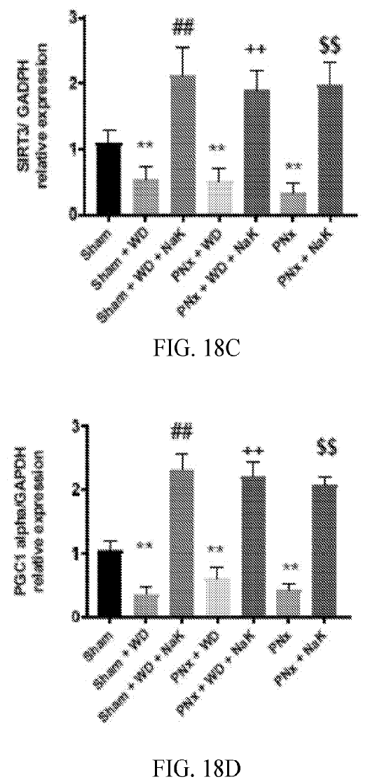

Lenti-adiponectin-NaKtide targeting specifically to adipocytes also attenuated inflammatory, apoptotic and mitochondrial biogenesis gene expression in adipose tissues of murine experimental uremic cardiomyopathy model. RT-PCR analyses demonstrated that, Lenti-adiponectin-NaKtide targeted specifically to adipocytes attenuated gene expression of inflammatory (TNF-α and IL-6) and apoptotic markers (Casp7 and Bax) in adipose tissues (FIGS. 17A-17D). In addition to the effects on inflammation and apoptosis, NaKtide targeted to adipocytes improved the altered levels of markers involved in mitochondrial regulation and mitochondrial biogenesis (Leptin, F4/80, PGC-la and Sirt3; FIGS. 18A-18D).

It will be understood that various details of the presently disclosed subject matter can be changed without departing from the scope of the subject matter disclosed herein. Furthermore, the description provided herein is for the purpose of illustration only, and not for the purpose of limitation.

Throughout this document, various references are mentioned. All such references are incorporated herein by reference, including the references set forth in the following list:

### REFERENCES

1. International Patent Application Publication No. WO 2008/054792, of Xie, entitled “Na/K-ATPase-Specific Peptide Inhibitors/Activators of Src and Src Family Kinases.”
2. International Patent Application Publication No. WO 2010/071767, of Xie, entitled “Na/K-ATPase-Derived Src Inhibitors and Ouabain Antagonists and Uses Thereof”
3. Wang, et al. “Involvement of Na/K-ATPase in hydrogen peroxide-induced activation of the Src/ERK pathway in LLC-PK1 cells.” Free Radical Biology and Medicine. 2014, 71: 415-426.
4. Yan, et al. “Involvement of Reactive Oxygen Species in a Feed-forward Mechanism of Na/K-ATPase-mediated Signaling Transduction.” Journal of Biological Chemistry. 2013, 288: 34249-34258.

What is claimed is:

1. An expression vector, comprising a nucleic acid sequence encoding a polypeptide antagonist of a Na/K ATPase/Src receptor complex, the nucleic acid encoding the polypeptide antagonist operatively linked to a promoter for expressing the polypeptide antagonist in a specific cell or tissue.

2. The expression vector of claim 1, wherein the polypeptide anatagonist comprises the sequence of SEQ ID NO: 1, or a fragment and/or variant thereof.

3. The expression vector of claim 1, wherein the nucleic acid encoding the polypeptide antagonist comprises the sequence of SEQ ID NO: 5, or a fragment and/or variant thereof.

4. The expression vector of claim 1, wherein the promoter is selected from an adiponectin promoter, an albumin promoter, a melanin promoter, a vonWillebrand factor promoter, an alpha myosin heavy chain promoter, an SGLT2 promoter, a MyoD promoter, a glial fibrillary acidic protein (GFAP) promoter, and a synapsin I (SYN1) promoter.

5. The expression vector of claim 1, wherein the promoter is liver-specific, endothelial cell-specific, or adipose cell-specific.

6. The expression vector of claim 1, wherein the expression vector is a lentivirus vector.

7. A viral particle, comprising the expression vector of claim 1.

8. A target cell, comprising the expression vector of claim 1.

9. The target cell of claim 8, wherein the target cell is mammalian.

10. The target cell of claim 8, wherein the cell is a mouse cell or a human cell.

11. The target cell of claim 8, wherein the target cell is an adipose cell, a liver cell, or an endothelial cell.

12. A pharmaceutical composition, comprising the vector of claim 1 and a pharmaceutically acceptable vehicle, carrier, or excipient.

13. A method of treating a Src-associated disease, comprising administering the expression vector of claim 1 to a subject in need thereof.

14. The method of claim 13, wherein the Src-associated disease is selected from the group consisting of vascular disease, cardiovascular disease, prostate cancer, breast cancer, neuroblastoma, tissue fibrosis, ischemia/reperfusion injury, osteoporosis, retinopathy, and obesity.

15. The method of claim 14, wherein the Src-associated disease is cardiovascular disease, and wherein the cardiovascular disease is uremic cardiomyopathy.

16. The method of claim 14, wherein the Src-associated disease is obesity.

17. The method of claim 13, wherein the polypeptide anatagonist comprises the sequence of SEQ ID NO: 1, or a fragment and/or variant thereof.

18. The method of claim 17, wherein the nucleic acid encoding the polypeptide antagonist comprises the sequence of SEQ ID NO: 5, or a fragment and/or variant thereof.

19. The method of claim 13, wherein the promoter is selected from an adiponectin promoter, an albumin promoter, a melanin promoter, a vonWillebrand factor promoter, an alpha myosin heavy chain promoter, an SGLT2 promoter, a MyoD promoter, a glial fibrillary acidic protein (GFAP) promoter, and a synapsin I (SYN1) promoter.

20. The method of claim 13, wherein the expression vector is a lentivirus vector.


SEQUENCE LISTING (12 sequences)

SEQ ID NO: 1 - protein, 20 aa, Homo sapiens
SATWLALSRIAGLCNRAVFQ

SEQ ID NO: 2 - protein, 13 aa, Human immunodeficiency virus
GRKKRRQRRRPPQ

SEQ ID NO: 3 - protein, 16 aa, Drosophila melanogaster
RQIKIWFQNRRMKWKK

SEQ ID NO: 4 - protein, 8 aa, Homo sapiens
KKGKKGKK

SEQ ID NO: 5 - protein, 33 aa, Artificial Sequence
  TAT NaKtide Fusion protein (pNaKtide)
GRKKRRQRRRPPQSATWLALSRIAGLCNRAVFQ

SEQ ID NO: 6 - DNA, 12432 bp, Artificial Sequence
  lenti-NaKtide-adiponectin promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcttgctagtgtatgcaatggtgtcagcgtttggaagctgattatgggatggatccctggatatggcaatcactagatggtctatccttttgtcacagctccaaattttgtctctgtaactccttccatgggtgttttgttcccatttctaagaaggggcaaagtgtccacactttggtctttgttcttcttgagtttcatgcgtttagcaaattgtatcttatatcttgggtattctaaatgtctgggctaatatccacttatcagtgagtacatattgtgtgagttcctttgtgattgggttacctcactcaggatgatgccctccaggtctatccatttgcctaggaatttcataaattcattctttttaatagctgagtagtactccattgtgtaaatgtaccacatttctgtatccattcctctgttgaggggcatctgggttctttccagcttctggctattataaataaggctgctatgaacatagtggagcatgtgtccttcttactggttgggacatcttctggatatatgcccaggagaggtattgtgggatcctccggtagtactatgtccaattttctgaggaaccgccagactgatttccagagtggttgtacaagcttgcaatcccaccaacaatggaggagtattcctctttctccacatcctcgccagcatctgctgtcacctgaatttttgatcttagccattctgactggtgtgaggtggaatctcagggttgttttgatttgcatttccctgaaggtccctactatttatctaacattttgaatgcaacattctttcctttgtacatagtcatatgccatgtgcattagtaatctgctataatacagacccaacgatgagacgactcagacaaaagagtgtatttgtatctcttgtgacatctaggatataggggaagaaacactccacacagttatgcagggctctctgttttgttttgtttttaaagatttattttatttcatttattttatgtatataaaatacatgaaatgagtacactgtagctatactgatggttgtgagccatcatgtggtttctgggaattagaattcagggcctctgcttgctctagttggctccagttgctccagtctaaagatttatttattattagatctaagtacattgtagctgtcttcaaacacaccagaagagggcataggatcccattacagatggttgtgagccaccatgtggttgctgggatttgaactcaggacctctggaagagcagtcagtgctcttaactgctgagccatctctccagcccttagcctgctctcttatatcatctatgagcacctgtctatggtaacagcaccacaatgggctggaccttacctcctcaatcatcaagcaagaaaacgtcccataggcttgcccacagggcagtctggtgggagcatttccccaactgatgttctctccccaaatgacttatctgatacactggccttcggagaaaagcaagtcaacacctcattgtgatcccattttacccagtcagaatgtcataagaaaacaaataacattttcataagaaaataaacaacaatacattctgtcaaggatgtagacagatgggatgcacttgattcactgctggtgggaatgataactagtccaatctcagtggaagtcagtctggagtgttctcagaaaatgaaaaaatagctgggcagtggtggcagatgcctttaatcccagcacttgggaggcagaggcaggtggatttctgagtttgaggccagccttgtctacagagtgagttccaggacagccaaggctacacagagaaaccctgtctcaaaaaaccaaaaaaaagccggacgtggtggcgcacgcctttaatcccagcactcgggaggcagaggcaggcggatttctgagttcgaggccagcctggtctacagagtgagtttcaggacagccagggctacacagagaaaccctgtctcgaaaaaccaaaaaaaaaaaaaaaaaaaaaaaaggaagtgaagaatagctgtggataacacagcttttcttttctttatatttcaggcatcagggatagaggtcaggtgtcaatgtaaatcactctaccactgagttacacttctaaattaaagacatttttcctttcttgaatgcccaaaaatttggagtactgcaggacaggctcactcttctgcagttagtgttatgtctcaaaatttgatttgaggtttagttgtttcagtaaattgtctttttaaagtcatgactaaacaataatatacagtgaaatttaaccacattcaggaaacactgaggaaactcaggcctgtggggaaatctggccttttgtttaagtatctcacttcgcatgtaggcgagaattgagatgtgaacatgagctcctgaaggaaaggaccacgtttctgtggctgcttcactctgggcagtaacacatctgtccaggtaaacccagccttgctgaacgtcaatctcagcacttgggaggcagaggtgagagtttgaggccagtctgatctatagagcaagttccaggatagcttgggctacactgaaaaaacactgtcttgaaaaacaaaacaaaacaaaacaaaacaaaacaaaacaaaacaaaacaccaaaaaccaaaaaagaaaaaaaaaaacaggaaaagaaaagaaaaaaatcaaaataaaacaaagccaaaaacaaagcaaaaaaaaaaccacccagcaaaaaaccaaaccgcctagcctcaagacatgtgtggttgaatgtttttcacttctagtcgctaagcaagtgtgtgtttttacacaatgccctctgtggtgagtggcggattcccctgagagttcaccaaatgataggctttcacaatgctcccgggtgtctaccagacccagcaaagtattgatgtggttttggggtgaaagtcactctgtcttgtgcaatagttagaatctgctgaaaccagcagtgttcctatatgggacaggggtccagagctaacccggaggctataactgagcagaggtgaagaccacgaggcattggggagcgtatgccctttgtggtcagagagatctagcttcgtgccttgggtctgtgtctctccctcttactggcttctggcttcttcattaagtgggagacaaccacaggtatctgtatgggaagactcgactaccccttgactcaacattgcttgttacttactttgtacaagatactacttagtctaggggttatggagcataacctcaagtaggtaaagcccctgctccagcgtgtttgcattccagtaagaagcgaaagacagtaacacacatacaaaataagtaagaaaatgcaacaacagcaacaacaacaacacacacacacaaagtaagcaaaacgctaagggaaagatagagagtgatacagctttgagttgctgtagttcttctctctcctttgcttcatacagtttgcttgggaagtgtccagggccatggggtcacaactaacagcccttggaaatgagcttgtgtccttaatcttcatgacctaacgtgatttctctagaaacatcagtgcattaacaggaagacaagatggaagatcatattttggctctccttccttggtgggttgacactgctggtcctatccactagtaaaagcatgactcttaggctctgtgtggccagtggaaggtggcagttggaggaagcagatgcttggccagcctttgcctgggagcagtctagctctgagtgtcttattggagcagctgctggcatccagagttctttttggattcacgatttaattcaaaagctttgtgctcccgagaatcagctctggtctttcaaaaataagatgtgagtccgccgagaggctcccaaggtattgccttgccaactgcaagccttttaggagcagtttagtgagtggtgactgctagttgcagttggctgttagcccagagctaataatagatagaaaaggtatatacttaaggagtctggaaactgaggtttatctactcacagaaaatgagtttctaaaaaactagcttgaaacttacccagaaaaatcttagaacatggttctccaatgtcaaggtaagtgttctgtgacactgggcttgaattatgtagggaccacagattttagaatttggacccctgaacttgcttcacaccccaccaggaaccttcctgtacaacagccctcagaattcatctacatggtcttttctcagtatgggatccggtctagcaagtggagcacaccttctattgcttaaagatttgtttatgtatatgggtattttggctgcatgcatatttgcacaccaaaagaaggcagcggatcccatggaattactgtgggtgctgggaattgaactcaggacctctggaagaatagccagtgctcttaaccactgagccatgcctgcagtccatctattttttattctagtacagcccctcttcattcttactgaaatagtaatgcctgaaccacacagcttcacatttagttacaaagaaagagtgggagtatcatgtgacaattagtgttgttgactctccaggacaaacttatgggaaagggaggtctcctgacccctgaacaatcattttacttgaggataattttcattgcactcagaaacatgctgaattattgtccttacccttgccccatctcttgctctggtagagaatggccaaagcctggaaacaggatggcttgacagaagctctacttggcttcccagacccaagctggattaaaccaggttccctaaggagtcttaaggcagctgccaggagcaaggggcccactcattggctattggccttgactgggttggccaatggtaagctggggtctgcctgtccccatgagtaccagactaatgagacctggccactttctcctcatttctgtctgtacgattgtcagtggatctgacgacaccaaaaggtaagaacaattctatattctcggctggctgggtatgaatgcagaatcccacttgggctgtgttcagattttgctctctgcagcagtgtgaaggtagatgttgagaaaactcaggccttggaaacatggttggggcagataagctttggggcttttctttaactcttcaaagctctaagaaagaaacaataacctttcggaaccccaactaagacactgatgaagacctcctgggagagtgagggctgggtagccactgaaggctctctgggagaggcgagtatgtagatgcagatctttggagtggattccacttagctatataggacatgatgcaggtcctgattggatgtgccatgtgagtctgcctttcccatgactattcacctgtcaatttcagggctcaggatacaagtttgtacaaaaaagcaggctgccaccatgagcgccacctggctggccctgagcaggatcgccggtctttgcaacagggccgtgttccaggagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 7 - DNA, 10695 bp, Artificial Sequence
  lenti-NaKtide-albumin promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctagcttccttagcatgacgttccacttttttctaaggtggagcttacttctttgatttgatcttttgtgaaacttttggaaattacccatcttcctaagcttctgcttctctcagttttctgcttgctcattccacttttccagctgaccctgccccctaccaacattgctccacaagcacaaattcatccagagaaaataaattctaagttttatagttgtttggatcgcataggtagctaaagaggtggcaacccacacatccttaggcatgagcttgattttttttgatttagaaccttcccctctctgttcctagactacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcactgtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagaaagggtatagatataaaaattaaaactaatgaaatggcagtcttacacatttttggcagcttatttaaagtcttggtgttaagtacgctggagctgtcacagctaccaatcaggcatgtctgggaatgagtacacggggaccataagttactgacattcgtttcccattccatttgaatacacacttttgtcatggtattgcttgctgaaattgttttgcaaaaaaaaccccttcaaattcatatatattattttaataaatgaattttaatttatctcaatgttataaaaaagtcaattttaataattaggtacttatatacccaataatatctaacaatcatttttaaacatttgtttattgagcttattatggatgaatctatctctatatactctatatactctaaaaaagaagaaagaccatagacaatcatctatttgatatgtgtaaagtttacatgtgagtagacatcagatgctccatttctcactgtaataccatttatagttacttgcaaaactaactggaattctaggacttaaatattttaagttttagctgggtgactggttggaaaattttaggtaagtactgaaaccaagagattataaaacaataaattctaaagttttagaagtgatcataatcaaatattaccctctaatgaaaatattccaaagttgagctacagaaatttcaacataagataattttagctgtaacaatgtaatttgttgtctattttcttttgagatacagttttttctgtctagctttggctgtcctggaccttgctctgtagaccaggttggtcttgaactcagagatctgcttgcctctgccttgcaagtgctaggattaaaagcatgtgccaccactgcctggctacaatctatgttttataagagattataaagctctggctttgtgacattaatctttcagataataagtcttttggattgtgtctggagaacatacagactgtgagcagatgttcagaggtatatttgcttaggggtgaattcaatctgcagcaataattatgagcagaattactgacacttccattttatacattctacttgctgatctatgaaacatagataagcatgcaggcattcatcatagttttctttatctggaaaaacattaaatatgaaagaagcactttattaatacagtttagatgtgttttgccatcttttaatttcttaagaaatactaagctgatgcagagtgaagagtgtgtgaaaagcagtggtgcagcttggcttgaactcgttctccagcttgggatcgacctgcaggcatgcttccatgccaaggcccacactgaaatgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaacccccaagtttgtacaaaaaagcaggctgccaccatgagcgccacctggctggccctgagcaggatcgccggtctttgcaacagggccgtgttccaggccacgaacttctctctgttaaagcaagcaggagatgttgaagaaaaccccgggcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 8 - DNA, 12496 bp, Artificial Sequence
  lenti-NaKtide-alpha MHC promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattgcaagtttgtacaaaaaagcaggctgccaccatgagcgccacctggctggccctgagcaggatcgccggtctttgcaacagggccgtgttccagggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 9 - DNA, 9669 bp, Artificial Sequence
  lenti-NaKtide-SGLT2 promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgactggacatcacatggtaccaaacaaaagacccagtaccaggaatgagttaccatcttatgaagtcattaaccatagagagctaatggtagcctcaatcattacaaaagctgttgccaaggttactgggtgctctcataccctgatggtaagaccctatggctgaagatggaacttattgatatccttgaacattgagaaattgagctgtgtgactagaagcttcaccccatcgacgatggtcacagtgctgcaaagtgctatgttcaggaggaaggtatccagccgcctccctcagccacactctgagactcacaccagagacctgctcaaagggcatgcccactggcccacaaatattatgggagcaactgaccactttttgggggtgtatttaaggtccacttcatgaaatgggacccatccctgacactgctaaaatgcctgagaacctgaaactagatagagcaagggctctaggggaaagctcactccagttattctaagggcacagggttatgacgcctaatgacatatcgctggctacatcccttggccagcacatcactgaaacctcaccagaggagcttcttggagtagaaggtgattaacagaactgtccgtgactggatgacttgcagagagtgagagacttgggagcactcagacttcaatgggatgcttgtatctcacccctctgctaaagatgcaggttctatacaggaggtgcaaagattgtaagagtcagagttggaggttgtcttcaggaaagcagagttttgcagacacaacaaagctgatgaaaatctgaactcacagacagtgatagcatgcacaagacagatatctgaactcacagaccgtgataacatggcacaaagacctgcacaaagttcgaaccaaataaaatctgagcatggaaaatgaggtgtgggcacaaagtcccactcctaagtaagaaactacttgcagttgacagctactaggagagagaaatgggttttcttcaatggagagacactgggtgtatcaactgcaccccaaggcaggccacacgctcaggaagagttggccaacacaaaacacactccgtgtttggttttctgtttgcttgggtttgtttttgtgcttttattcttccttcctttctttctttgtttctttgtttctctctctctttctctctttttctctctccctcctcctcttcttctttcttctttttcttcttcttcttcttcttcttctttttcttcttcttcttcttctttttcttcttcttcttcttcttcttcttcttcttcttcttcttcttcttcttcttcttcttcttcttcttcttcttcttctgatcagagaggggaggggatctgggaaaagtttggggagaggaaagaatttgcccaaaatatattgcataaaaactttttaaaaataaatttaaaacaatttttaggatagagcaaagagaaagtagaaaatatttgggttgggaagggcaggagaatgagggaaatgtgatttttttctccctaggttttgtatggcagaagtcagggcatggcatgcgtgacagtcaggctgaggaacatgtatgtcctgctgactgtcagggggtgctatggaggacttgtgcggaggacactgtcagagttggattcggaccttcctaatcaaagttagagggtgtattttcagagaacgcaggaaggaactttgcttggaacactgggtataggatggatcctaaacccaggaaggagtgctcttgaattccaaatggtccagcgccccaggaccagccttcggccttgatagatcctgattcagataaataaagctggagaaggaggctgagacctgggggacttgtcgggtcagtgctcctgaggtaaccattaatccttcccccaggggaatccagggactagccccttgagggacagatggtggagagaattcagcaacacgtagaggcaggctctgaacttggggagcagaaggtcctgattgataatcctgctgacattctggttatcgctgcctatttcctgctggtcattggtgttggcttgtgggtgagacattgaggggggttggatagggaaatgcttctggggcttgagggtaaagatttagggagacctcagagaggagtgggagaaaagggtgcttggatataatgagggagaaacctagatttagtaggcaagccaattttaattctttgtcttcgtaccttctggattgtgcaaaagagactgggggtatcaataggtttttttttaattcaagtgttctaacaagtgctctaagagatgtatcagttcccacgtctgtattatggctgagcagcagcctatatttaaggtcaccaggcaagttaggctgaatctaggcatatctaggttccagtagttgcgctaggattagggcctgggttgttctgagtgtcggggaaggttgggggtaaggaggtgcagtctggggagtccagggctggttaatcttcagcctgaaacaaggctgaggaatgtgttgaggaagctaaggaagtccaaagatgtgccccaatcccagtttccccccacttctgtttcccagtctatgttcagaaccaatagaggcacagttggtgcaagtttgtacaaaaaagcaggctgccaccatgagcgccacctggctggccctgagcaggatcgccggtctttgcaacagggccgtgttccagggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 10 - DNA, 13103 bp, Artificial Sequence
  lenti-NaKtide-MyoD promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggggcccagccacaggatggtatagtatagaatagattttatttagagcatagggaggggagtcaagagggcagtagaggcaaagaaaggaagagagagggggagagaagagaagtagaggtgtagaggctggccttggagcacgtggagagagcagggggaaaggaatggggagagaggggagaattggaaacagagaaagagcaagagaggatcaagagagcaaggaggggggcaagcagccccttttatagtgagtcaggcacacctggttgttgccaggtaactgtggggcggggcttagacacaaagctaaaaacatccaccacctagttcatctgccagactctcaaggtcctgcacataccagagcctgggggaatcccagagggagacatgtgaaggctccctgggaccctggtcaggaccaggaccatgtctgtgcccagccagagttcctctaggtcagcctaaattggccagatctacacttggtggcaggtagtttcaggctttctgggaagcaaaactggcagagaacagagcaggatccttgagttgggaaaggaaagtctagggccagagactgaacctggggctggtcctgttccacctgtcctccccgtggtttcatcctccagtccttcagccccctagacccaagccagccatgcagcccgcagtagcaaagtaagaggccacaggtccagactgggtagggcagaggtgcctgaggcttggggcaggtgctagttggatccggtttccagaggctatatatatataaaggctgctgtttccccatggtgcaacaccccagaggcctagccagaccaacattcctgcccaaaagccagctctccatttatagcaccttggaagactagccaagggagctgaaatgcaaggcctggaaaggacagggggaaatcaaaggggccacctatggcggcaggagaactgagcctcaggatgagctgtgtgcttctccaggtcagtgggcctacagcctaagaggccctgcattgaggggacaatgcctcagcccagagccaatggcacgctccagaaggggtggctgggggaagttttagtgaccataaaataaaaagcaaggttgcaatcacttagactcagcataaaatttatttcggtttttgttatttgtgtgcttgctttgctttgctttgtttgctcgggtttgagataagagagagagagagagagagagagagagagagagggaaggagggagggagggagggagggagggagggaggaaggaaggaaggaaggaaggaaggaagaaagaaagaaagaaagaaagaaagaaagaaagaaagaaagaaagaaagaaactagatttgcacaagtggtttgaagtgcttccttggagacaccagggcactgcataatgaaacagacttgctcattcatccgccaaggaatatttgtggtgcacctagtgtgtgctgagcatattcagctgaggttccaaaaaggaggaagcaatagcttccatcgaaatcattactaacgacaattacatccatccctcactgtagatttaagtcaaatgaaagagcacttatgatgaaccccacatccatcggcagcatactgttggaatgttgcaaccgaccaatgggagagagcacgtcccaggcaaaccagcctctgctttgcctgggcagaggcagcgtgagagcttgtatgagtaagtacctacatagagcccaggtttgctggaatagaatgacttgtagcacattttgctaaattcaagtataaggatagaaatcaaaagagcccaagactgttcattcactcgcttgacacttaaaccactgtgcccgccgtgggatcactgctgcagtggcttccggagacgccatggtgagcaaagtactcctatccatggtatgctggtcttcgtgtccctcggtgaataactgacaagatcattctcgaggaaaggctgtgtgacattccccaagcctgggagacatctatctgggaggaaatggccagagacatcccaaaaatgactttggagttgagatctgagacctgaaaagctgccagtcacaggcaggaccaggggagagggcagcaggcttgggacagcaggcacgagacatcatggctctgaaggaggctgtgcagggctgaacgaagggaaggtggctaaccagaatagggctatctgaggggacagtgtctccaggtgacattgtggagacagggtgagtagggttcctgaatggccacctagttggagaaggacacgaggtttgcatctggcccaatgggaagcaccaggaccgccatcattccctgaggctgccgtgcagggactgggttccaggagggacaaatgtgctagagagtgtcctacccattcctctccccctccccctggctcctcctcccttgaggcatgatcagaccagaggcccgagaagggaaatggaggcaggaacagatggaggtgatggggacctcaagtggtgggtgagcgcagcaagagacagacttctgtggaggcaggcttcagtggacagctcctttaactgaattaaagggtgtttagatttggaggtgagtggaggctatggggtgagtggacaccactggcctgggccagggtgctggtgctcttatgcctcatttgcacaaagatgaatggcaggtgctgcaggatgagacatgaccttataaggagaaagtagaaggctgtctggctatctgacctcctctgctggggcataggtgggggcatggatctatatgccctgggacatccaggcccagggcaagaggtggggaggagtcctattcctgccggtttcaggatgagattttcagggtttggaaacactttgacccgactcttgctccaggcctactctctgtggctctgtggttcccctggccccacaagagagggtaaaagacagaattagtggggccatctaagtcagcaaatggcaggagttagcatagtctggatggtgaccttagaaatggcagaccttggaggtgtcttacaggaacagtaggcaggacttcatcatgcccagccttcccctcgggacctctggccacatgaatcccactcaggctttacacttccacctgaatgtttattgtgaatttcaaattttgctcaaactggatctttgatctgatactcacagacccactatgccccagcccctttcatccaaaagcgatctttaatgtccctcttgtccctgtgtccacatccaatcataaagaaatcttgtttgctccatctctacttttaaagatttaaaagtttttatgtacgtgcgtgctttgcccgcatgtaagtttgtgacgcacaaaggtcagagagggcatcaggtccctagagctggggttacaggaagttgtgaacaaccatgcaggcactgggaatcaaacccaggccctctgcaagagcagtgggtgcccttaactgttgagccatctcacctctccgagcccacccaaccccatcttgagagcatacctgcaactgaactacttctcaccagattctctttgcctcccaatgctaaacaaccatctgagtggccatgagcacccatttggactgtggcagtgccctccaaatggatcacctttctgtccttaccttccacgtacctgtaaaatcccaagctgagacctgatacagccaatcctagggcaagttcaggaaagaggactaaagctgagagcagcgagataactctgaggctgaatccctgtttagtgttacaggaagaggagaaagagggggaagaagaagaggaggaggggggaggaggaggagaactggaagaaaaaactagaatggagccattaagaagaatggtggctctcagtcaagctgggggggcacaagctgagtttgagtctacttgctgtgcattaaagcagtcaagcacagtcctgtcccaggacctttgcacatgtttgcccttctctctaactctaaatacctgctagaagtagcttctgtctcccttcatccagggcactacacgaacacaaccatcccagaggaaccttccctgattgctggaatagagattattatttttttttgctttgttttgttttattttattttatttcatttcattcatttcgtttcgttttgtggaatttgtaaatctctgacaccacaaagttccttgcttgattgacctacctataattcaactatgtgcatagggccttggttgcgcttactgctgggttcccaatacctggaccagtagctggtctgtgatagataagccatggtgaatgctgaatgaataaacaaagtgaaaccatggtgtaaagtgtaagcacttcatgatggggtctatcagcaccgccagtatcagagacaaaaaccgttggacttcaaaagaggttcctgggtgagtctgatggatagagagggctttccagtttgtaaaaaggaacagttaaaatggcttccagaagttagtgggaaagacacctacgtcacccttgcttggattcctggcatccgggagagaagacatagcaaaagtccatagccaaggaaggcttctagccagagccccccaaaagcatgaacatcccagggttggggtgtgttgggatgtgtaggagagccagagatcccacaaggtggctccttccttttgatcttacctccccagttagcctgctggaagagacccagctgaagtctccagaagaacagagccagagcccccagagccctctgccagatctcagtgctgcaggcaaagccagagaaggttgctggttatgctatgcaagcgggcaggcaggtgctggaaacagaagcaagtctcccacctccactgacatttccacatccatccctggtttggattctcacccatcaccacagttaccccctggacattgtcattgttcctggatatgaagtggatacagacttgggaagaggtcatctcaacaaagttatgccgactctaccaactctacagtaggtgaggttgtcaggccaggttccagccatcacgatccttaagaaaggcatttcccagggttcactcagagcaagatggcacaaacccatagcaaacaggttttcttcttctcggtagaaacagatcaacctagacccccatgggtctctagacactcacagaaagtaagttggagggagcctccctctctctccctccctcttttccttggactgtccacccggagtttgagcagaatggcttgaagttttaatgatgattcccactacgcatgcaaggacagcgctggggttctaagcttcccgtggaagaacagatattctctgagccattcccagatggagaatgaccaaatagcactgccaccgattcatttgtgccaggcttgctcacctagaccttctgagtctcactgtctcttgcccactttgtccttggctcaacttctctgggtgtgtcatcatttctccactcttctctagaactttcattgtcccgtagccttgagtctctctccaaacctcctgcaatctgatttctaactcctatgctttgcctggtctccagagtggagtccgaggtcagctccgaagtgagcactgaggtcagtacaggctggaggagtagacactggagaggcttgggcaggctgcaccagatagccaagtgctaccgcgtatggctgccagtctctctgccctccttcctagctaggcagctgccccagcacagagtcgcgggagggggcactccctggccccagtggctaccctggggaccccaagctccgccctactacactcctattggcttgaggcgcccccgcccccagcctccctttccagctcccgggcttttaggctaccctggataaatagcccagggcgcctggcgcgaagctaggggccaggacgccccaggacacgactgctttcttcaccactcctctgacaggacaggacagggaggaggggtagaggacagccggtgtgcattccaacccacagaacctttgtcattgtactgttggggttccggacaagtttgtacaaaaaagcaggctgccaccatgagcgccacctggctggccctgagcaggatcgccggtctttgcaacagggccgtgttccagggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 11 - DNA, 9249 bp, Artificial Sequence
  lenti-pNaKtide-GFAP promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggcaagtttgtacaaaaaagcaggctgccaccatgggcaggaagaagaggaggcagaggaggaggcctcctcagagcgccacctggctggccctgagcaggatcgccggcctgtgcaacagggccgtgttccagggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 12 - DNA, 7540 bp, Artificial Sequence
  lenti-pNaKtide-SYN1 promoter
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgggcaggaagaagaggaggcagaggaggaggcctcctcagagcgccacctggctggccctgagcaggatcgccggcctgtgcaacagggccgtgttccagggaagcggagagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt